(19) United States
(12) Patent Application Publication
(10) Pub. No.: US 2005/0269563 A1
(43) Pub. Date: Dec. 8, 2005

(54) Fullerodendrimer-comprising film

(75) Inventor: Yutaka Takaguchi, Okayama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: ECODEVICE LABORATORY CO., LTD., Saitama (JP)

(21) Appl. No.: 11/199,190

(22) Filed: Aug. 9, 2005

Related U.S. Application Data
(63) Continuation of application No. 10/383,695, filed on Mar. 10, 2003, now abandoned.

(30) Foreign Application Priority Data
Mar. 3, 2003 (JP) 2003-056307

(51) Int. Cl. H01L 29/08; H01L 35/24; C07C 321/30

(52) U.S. Cl. 257/40; 568/66

(57) ABSTRACT

A thin film comprising a fullerodendrimer and a method of manufacturing a thin film comprising coating on a substrate of a mixture of a fullerodendrimer and a solvent. It is possible to further incorporate an organic polymer and/or an inorganic polymer. The fullerene may be C₆₀ or C₇₀. A product comprising a substrate on which is provided a fullerene thin film. A method of manufacturing a product comprising a substrate on which is provided a fullerene thin film, comprising providing on a substrate of a thin film comprising a fullerodendrimer and decomposing of at least the dendrimer constituting the fullerodendrimer by heating in a non-oxidizing atmosphere.

### TECHNICAL FIELD

The present invention relates to fullerodendrimer-comprising films, substrates having fullerene films, and methods of manufacturing the same.

The fullerodendrimer-comprising film and fullerene film of the present invention can be applied to organic semiconductor devices utilizing the optophysicochemical characteristics of fullerene, and in particular, to solar panel materials, organic EL materials, photorefractive polymers, electrophotographic light-sensitive materials, film materials having environmental cleansing actions, and the like.

### BACKGROUND TECHNOLOGY

The fullerenes, denoted by C₆₀, have substantial capability as electron acceptors, and in particular, their application to photoelectric conversion elements operating by generating holes through the movement of optically excited electrons is greatly anticipated. However, the fullerenes have poor solubility, and their dispersion in polymers and processing present difficulties.

With the aim of improving the functioning of fullerenes, attempts have been made to combine fullerenes with dendrimers (for example, non-patent reference 1 (V. J. Catalano, N. Porodi, Inorg. Chem., 36, 537 (1979); non-patent reference 2 (Y. Murata, N. Kato, K. Fujiwara, K. Komatsu, J. Org. Chem., 64, 3,483 (1999); and so forth).

Non-patent reference 1 describes a method of constructing a fullerodendrimer as an enclosed complex incorporating a fullerene host molecule in a dendrimer. Non-patent reference 2 describes a method of constructing a fullerodendrimer as an iridium compound.

However, both of these methods require special compounds for the reactions and neither affords an inexpensive method of constructing fullerodendrimers. Further, components that can be employed in the dendrimer are limited, and neither method is suited to imparting various functions to fullerenes at will.

With the goal of improving the solubility of fullerenes and imparting new functions thereto, the present inventor conducted extensive research into developing a new method of synthesizing fullerodendrimers (substances in which a fullerene is bonded to a multibranching tree-like polymer). As a result, he discovered that the new fullerodendrimer obtained achieved high solubility while retaining the function of a fullerene.

Accordingly, the object of the present invention is to provide thin films employing new fullerodendrimers and fullerene films formed with new fullerodendrimers for application to organic semiconductor devices, including photoelectric conversion elements.

### SUMMARY OF THE INVENTION

The present invention relates to a fullerodendrimer denoted by general formula (1) or (2), and to thin films comprising these fullerodendrimers.

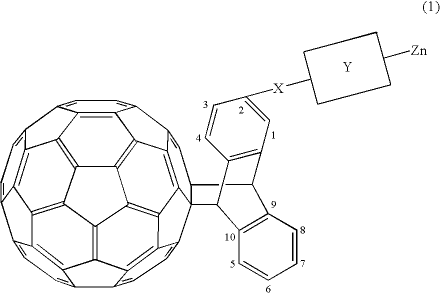

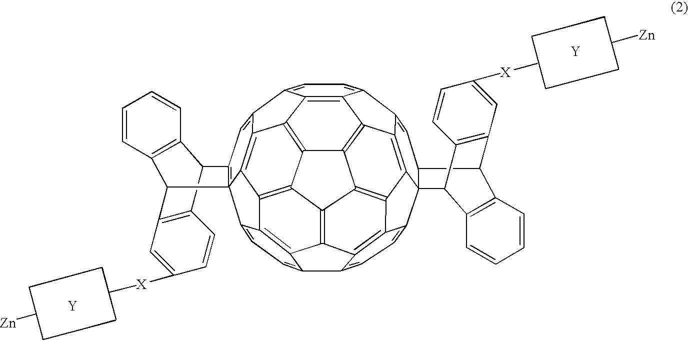

In the equations, X denotes an electron-attracting substituent, Y denotes a spacer, and Z denotes a terminal functional group required to achieve a function. The number n of Z incorporated into Y can be from 1 to 3.

The present invention further relates to the fullerodendrimer denoted by general formula (3), and to thin films comprising this fullerodendrimer.

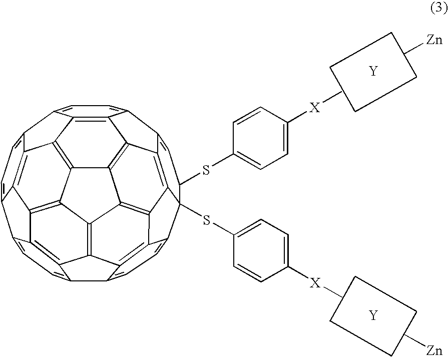

In the equation, X denotes an electron-attracting substituent, Y denotes a spacer, and Z denotes a terminal functional group required to achieve a function. The number n of Z incorporated into Y can be from 1 to 3.

[The Fullerodendrimers Denoted by General Equations (1) to (3)]

X denotes an electron-attracting substituent such as —C(═O)NH—, a carbonyl group, ester group, amide group, phosphoxide group, phosphonic ester group, or the like.

Y is a spacer examples of which are: alkyl chains, polyethylene oxide chains, and dendrimers (polyamidoamine dendrimers, polyphenylether dendrimers, polyphenylester dendrimers, and polyamide dendrimers). Specific examples of dendrimers are —CH₂CH₂N(CH₂CH₂C(═O)NH—)₂ and —C(CH₂CH₂C(═O)NH—)₃.

Z denotes a terminal functional group required to impart a function, examples of which are hydrophilic functional groups, hydrophobic functional groups, oxidizing and reducing functional groups, molecular identification functional groups, polymerizing functional groups, metal coordinating functional groups, and liquid-crystal functional groups. Specific examples are carboxylic acid derivatives, phosphoric acid derivatives, diphenyl selenide derivatives, alkyl groups, fluoroalkyl groups, alcohol groups, amine groups, dendrimers, bipyridine derivatives, phenanthrene derivatives, styrene derivatives, acrylic acid derivatives, cyanobiphenyl groups, methoxyphenyl benzoic ester groups, cholesteryl groups, sugars, DNA, ruthenium bipyridine complexes, porphyline, methyl ester groups, polyester oxide groups, diphenyl selenide groups, fluorooctyl groups, and dendrimers comprising these compounds at terminal positions (polyamidoamine dendrimers, polyphenylether dendrimers, polyphenylester dendrimers, and polyamide dendrimers).

In —Y—Zn, the number n of Z incorporated into Y may be from 1 to 3. For example, when Y denotes the dendrimer —CH₂CH₂N(CH₂CH₂C(═O)NH—)₂, two Z groups are incorporated. When Y denotes the dendrimer —C(CH₂CH₂C(═O)NH—)₃, three Z groups are incorporated.

Specific examples of the fullerodendrimer denoted by equation (1) or (2) that is employed in the present invention are given below. Compounds in addition to those given below are given in the embodiments.

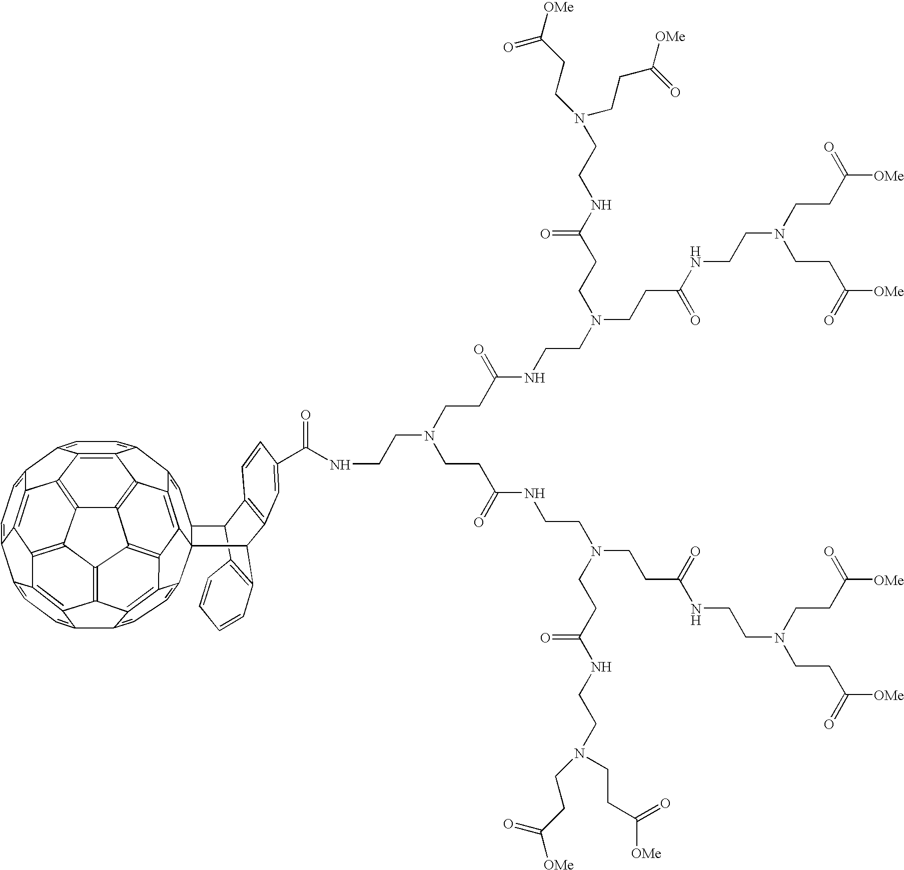

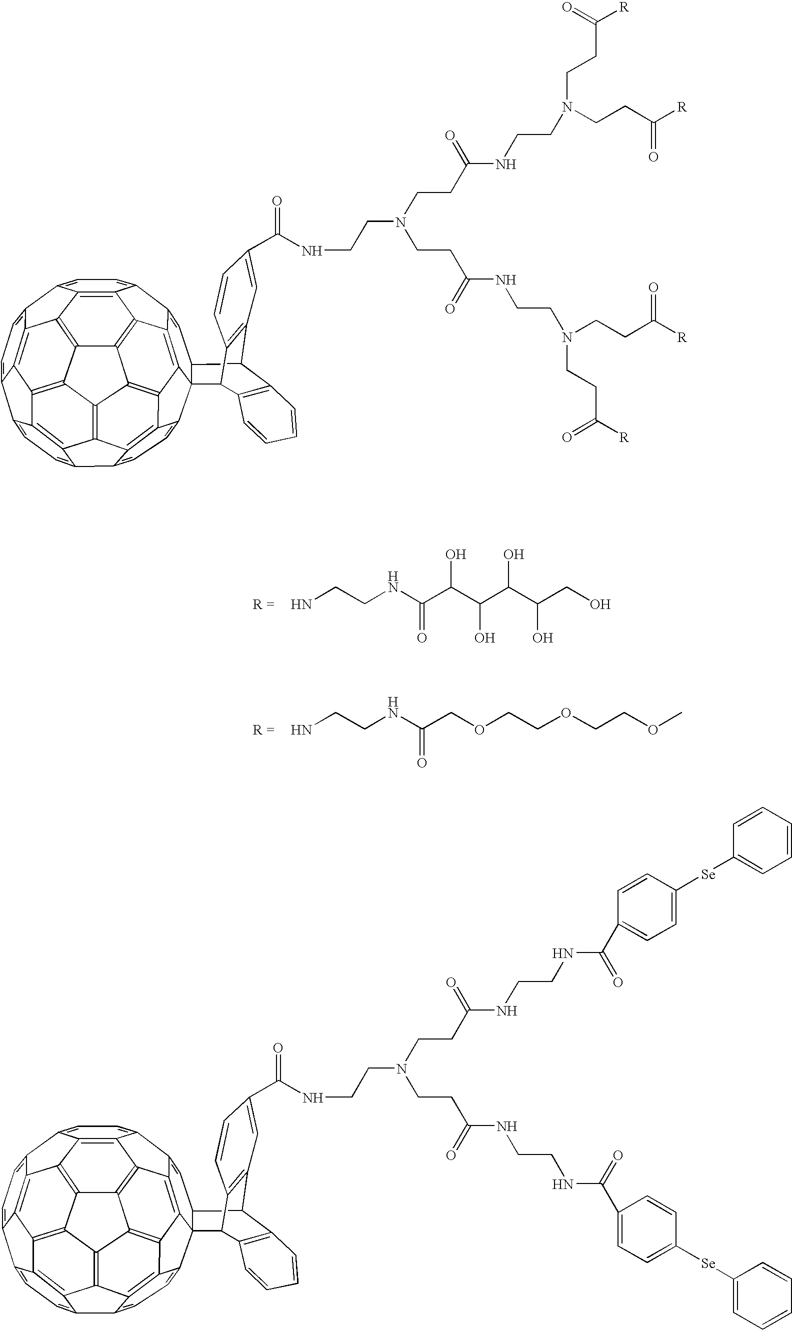

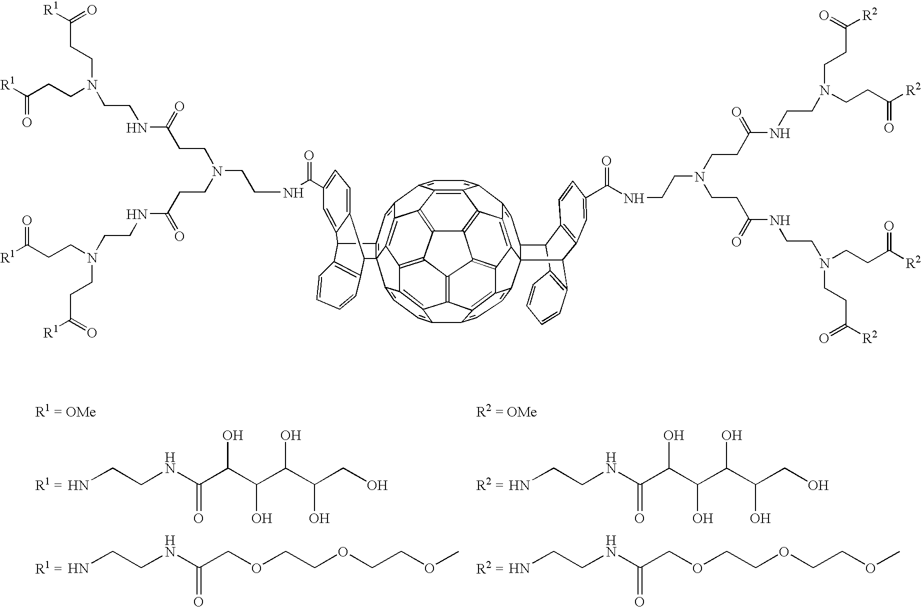

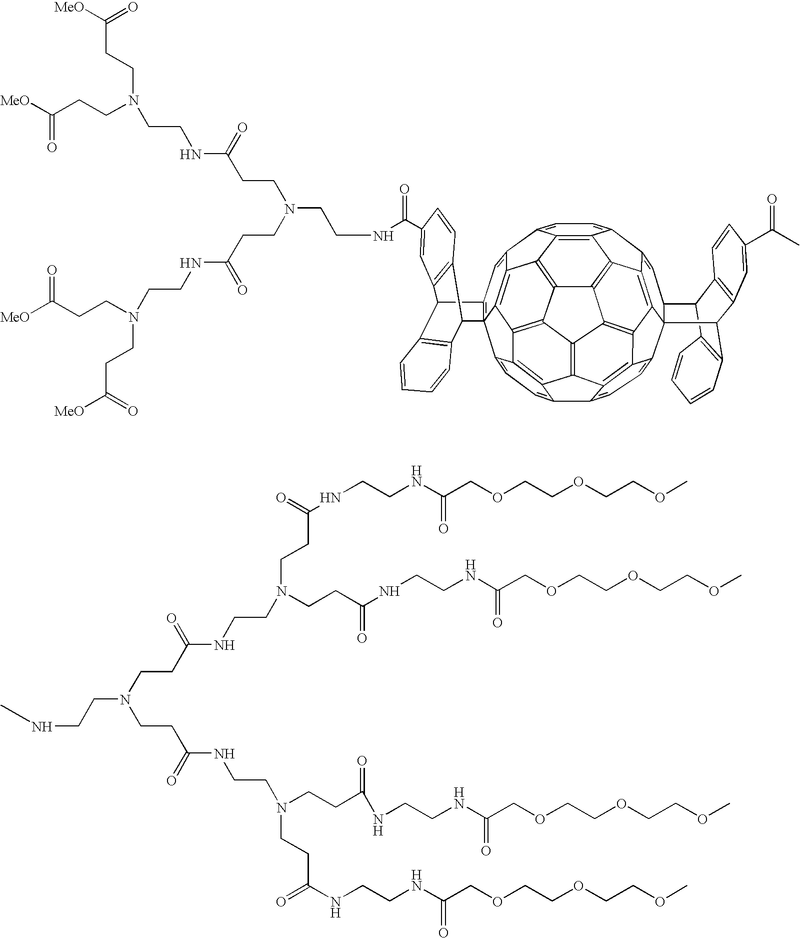

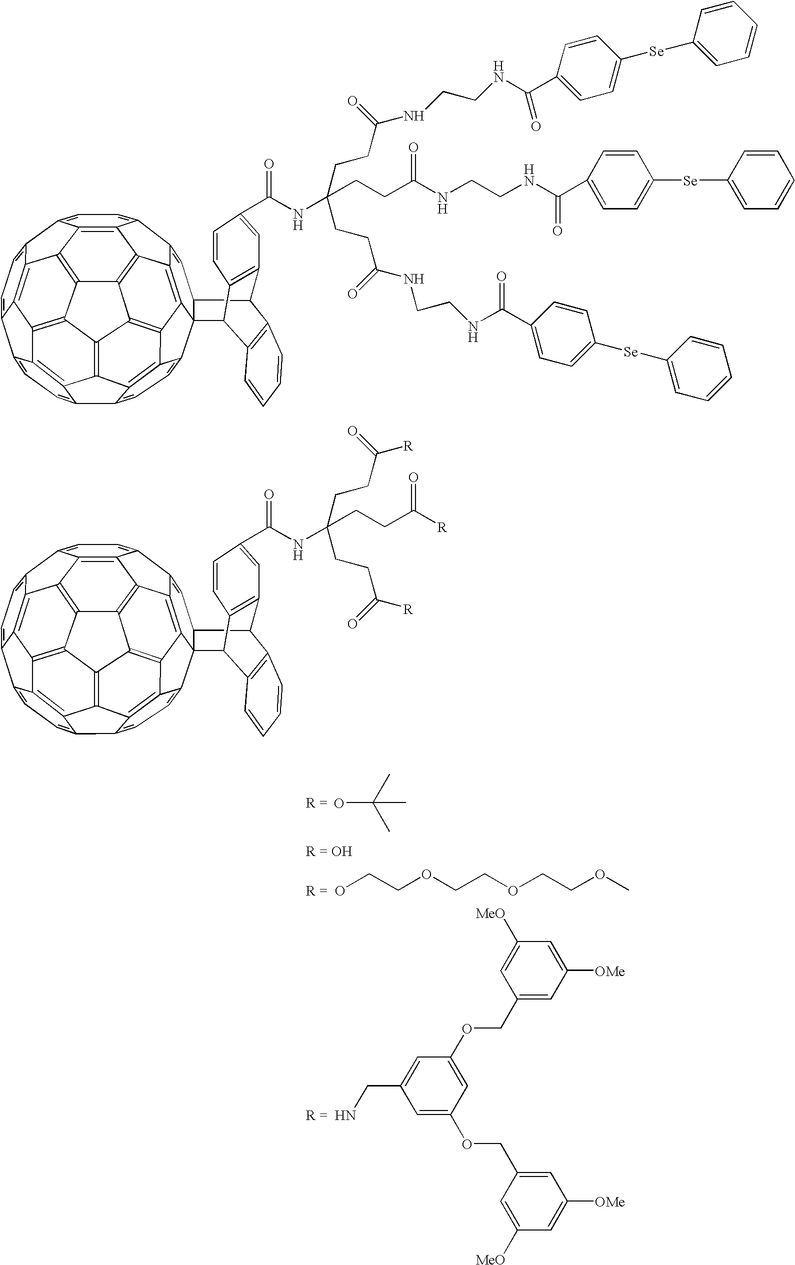

Specific examples of the fullerodendrimer denoted by general formula (3) that is employed in the present invention are given below. Components in addition to those given below are given in the embodiments.

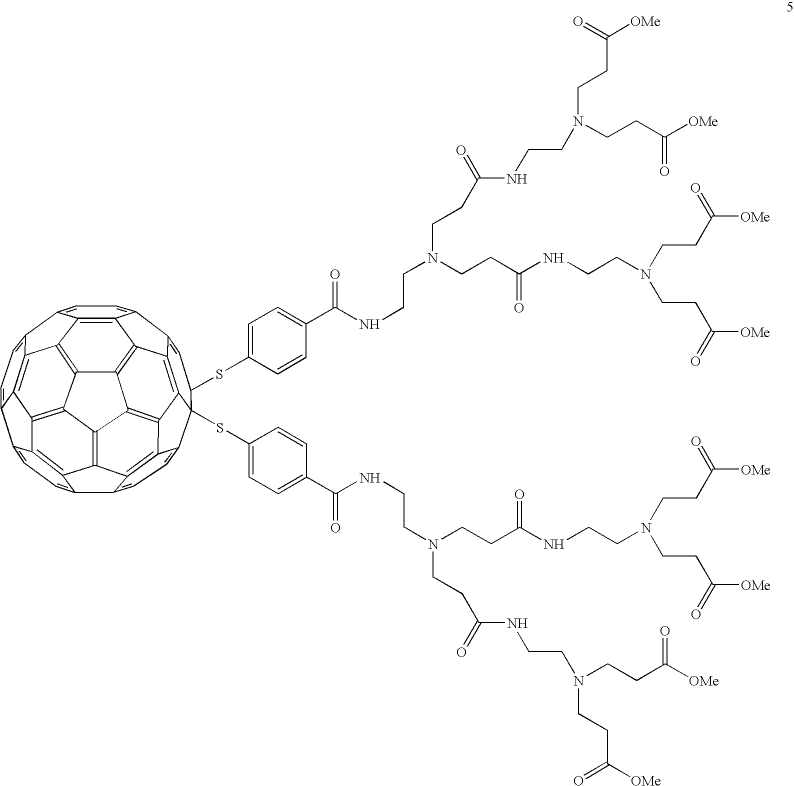

C₆₀ and C₇₀ are examples of the fullerene constituting the fullerodendrimer. When employing the thin film of the present invention as an organic semiconductor device, compared to C₆₀, with its highly symmetric molecular structure and highly degenerated energy level, C₇₀, with its rugby-ball shape and anisotropy, is often more advantageous for generating optical carriers. Further, for practical use in the important near infrared range of 700 nm and above, C₇₀ is reported to exhibit less of a drop in photoconductivity (Δσ) than C₆₀.

[Synthesis of the Fullerodendrimers Denoted by General Formulas (1) and (2)]

The fullerodendrimers denoted by general formulas (1) and (2) of the present invention can be synthesized by a Diels-Alder reaction of a dendrimer anthracene derivative and a fullerene.

Anthracene derivatives can be obtained by reacting a starting material in the form of 2-anthracenecarboxylic acid, for example, with methanol to obtain 2-anthracenemethyl carbonate. Next, diethylamine is reacted with the 2-anthracene methyl carbonate to obtain generation 0.0 polyamidoaminedendron (G0.0(NH₂)). When generation 0.0 polyamidoaminedendron is reacted with methyl acrylate, generation 0.5 polyamidoaminedendron (G0.5(COOMe)₂OFF) is obtained. Next, when diethylamine is reacted with the generation 0.5 polyamidoaminedendron (G0.5(COOMe)₂OFF), generation 1.0 polyamidoaminedendron (G1.0(NH₂)) is obtained. By sequentially conducting reactions with diethylamine and methyl acrylate, dendrimer trimer can be grown. This reaction is described in Chemistry Letters, 2000, 1388-1389, for example.

Further, terminal oligoethyleneoxidodendron is obtained by hydrolyzing 2-anthracenemethyl carbonate, generation 0.5 polyamidoaminedendron (G0.5(COOMe)₂OFF), generation 1.5 polyamidoaminedendron (G1.5(COOMe)₂OFF), or the like with an acid, and then reacting the product with oligoethyleneoxyglycol methoxide. For example, generation 1.0 terminal oligoethyleneoxidedendron (G1.0(oligoethyleneoxide)₂) can be obtained by reacting generation 0.5 polyamidoaminedendron (G0.5(COOMe)₂OFF) and HO—(CH₂CH₂O)ₙ—OMe, and generation 2.0 terminal oligoethyleneoxidedendron (G1.0(oligoethyleneoxide)₂) can be obtained by reacting generation 1.5 polyamidoaminedendron (G0.5(COOMe)₂OFF) with HO—(CH₂CH₂O)ₙ—OMe.

Further, perfluoroalkyldendron can be obtained by reacting acrylic perfluoroalkyl ester with polyamidoaminedendron. For example, generation 2.0 polyamidoaminedendron (2-G2.0(2-(fluorooctyl)ethyl ester)₄) can be obtained by reacting acrylic perfluoroethyl ester with generation 2.0 polyamidoaminedendron (G2.0(NH₂)).

Further, generation 1.0 terminal diphenyldiselenide polyamidoaminedendron (G1.0(diphenylselenide)₂) can be obtained by reacting generation 1.0 polyamidoaminedendron (G1.0(NH₂)₂) with a phenylselenobenzoic acid derivative, and generation 1.0 dendrimer (G1.0(methoxydiphenylselenide)₃) can be obtained by reacting generation 1.0 dendrimer (G1.0(NH₂)₃) with a phenylselenobenzoic acid derivative. For example, generation 1.0 terminal diphenylselenide polyamidoaminedendron (G1.0(diphenylselenide)₂) can be obtained by reacting 1.0 polyamidoaminedendron (G1.0(NH₂)₂) with 4-phenylselenobenzoic acid and generation 1.0 dendrimer (G1.0(methoxydiphenylselenide)₃) can be obtained by reacting generation 1.0 dendrimer (G1.0(NH₂)₃ and 4-(p-methoxyphenylseleno)benzoic acid.

Generation 1.0 dendrimer (G1.0(NH₂)₃) can be obtained by reacting 2-anthracenecarboxylic acid with Behera's amine (H₂NC(CH₂CH₂CO₂t-Bu)₃), hydrolyzing the terminal carboxylic ester to obtain carboxylic acid, and then reacting it with ethylenediamine.

Behera's amine (H₂NC(CH₂CH₂CO₂t-Bu)₃) can be obtained by reacting nitromethane (O₂NCH₃) and acrylic t-butyl ester to obtain the nitro compound (O₂NC(CH₂CH₂CO₂t-Bu)₃), which is then reduced.

For the synthesis of dendrimers using Behera's amine, see G. R. Newkome, C. N. Moorefield, F. Vögtle Eds., “Dendritic Molecules”, VCH, Weinheime, 1996, pp. 84-87. The description therein is hereby incorporated into the present Specification by reference.

For the fullerodendrimers denoted by general formulas (1) and (2) employed in the present invention, the example of the reaction between an anthracene derivative and fullerene in a Diels-Alder reaction is shown in the following equation. For example, the reaction is desirably conducted in a solvent in which fullerene is readily soluble, with the use of a solvent such as orthodichlorobenzene or chloroform being preferred. The reaction is suitably conducted at a temperature within a range of from room temperature to 60° C. for a period of about one hour to one week. The reaction product obtained may be purified and isolated by a known method such as column chromatography to obtain the target product.

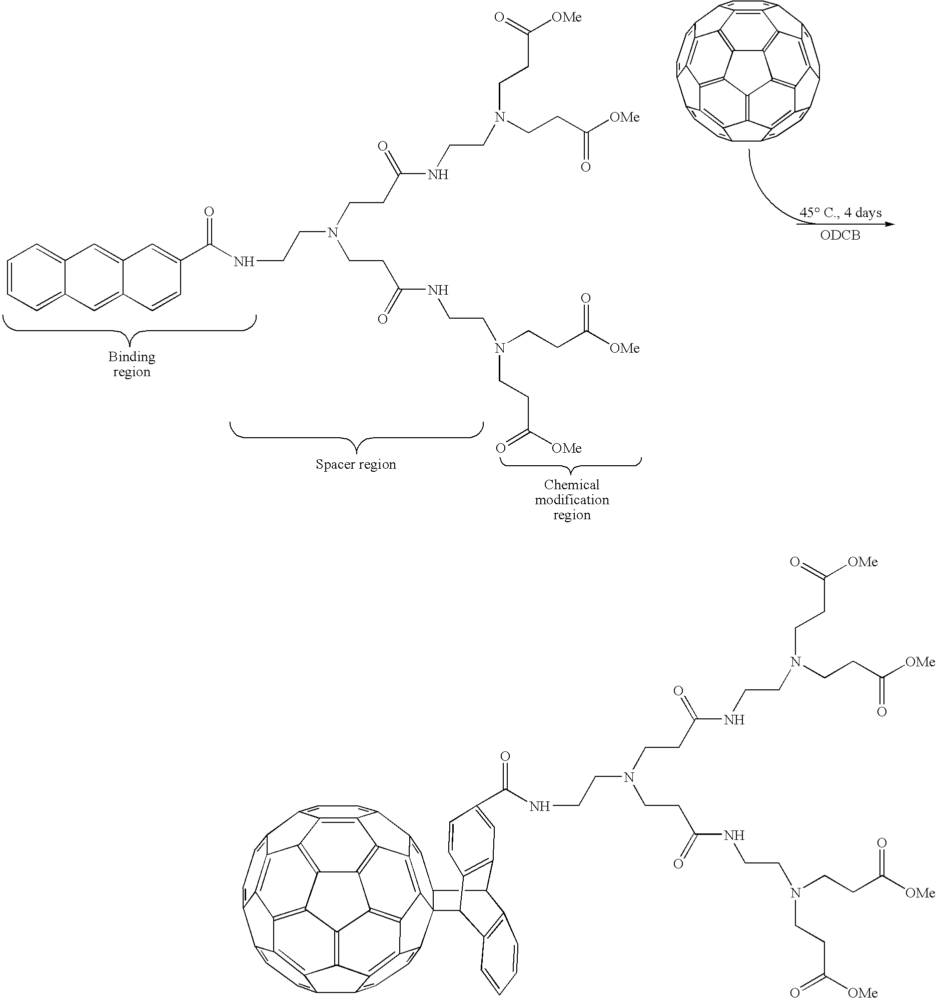

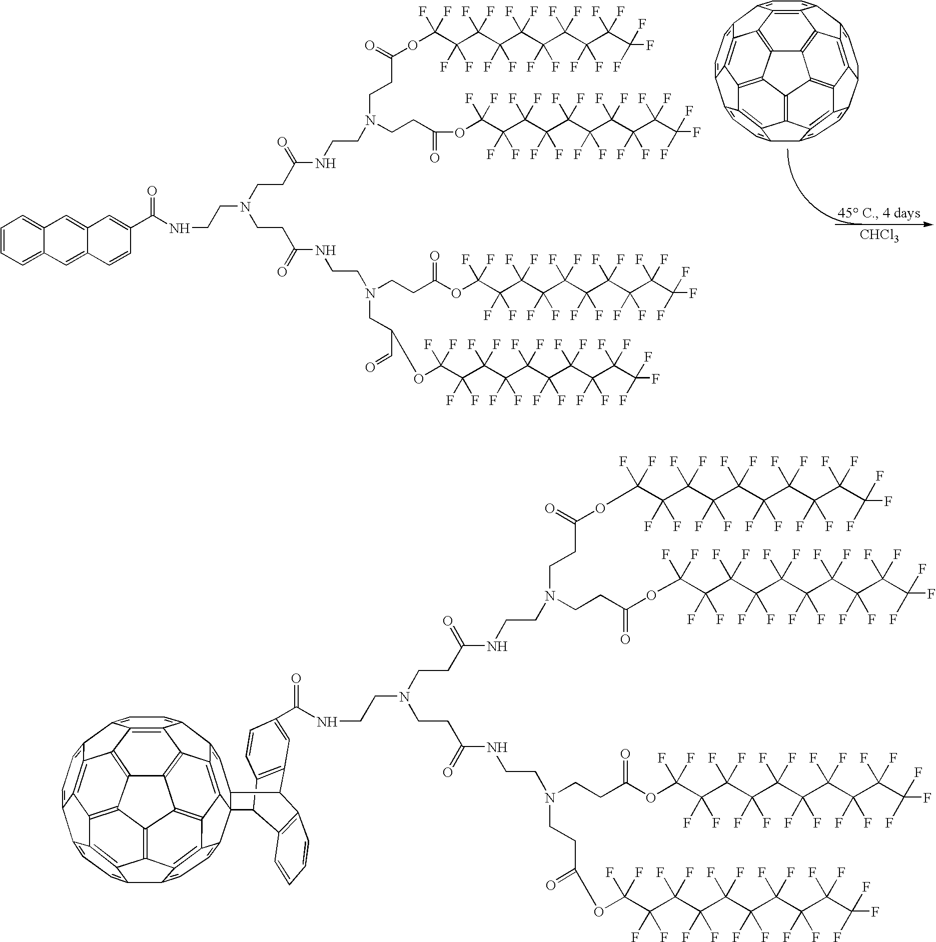

The following compounds are further examples of compounds employed to configure bonding sites. In the fullerodendrimer, the hydrogen in the carboxyl group or the methyl in the carboxymethyl group is desorbed and linked to spacer Y. 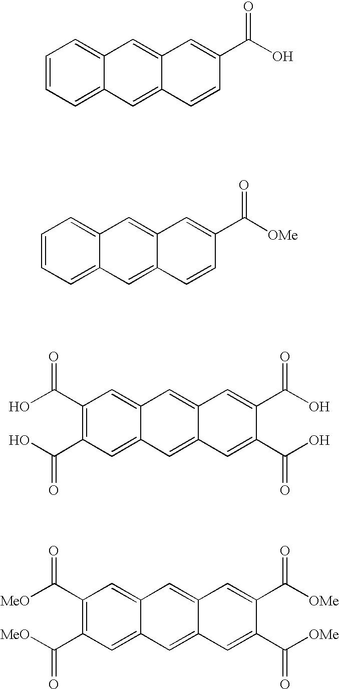
[Synthesis of the Fullerodendrimer Denoted by General Formula (3)]

The fullerodendrimer denoted by general formula (3) that is employed in the present invention can be synthesized by reacting a dendrimer disulfide derivative with fullerene employing diphenyldiselenide as catalyst. As will be described in detail in the embodiments, the dendrimer disulfide derivative may be prepared using 4,4′-dithiobismethyl benzoate as starting material, and in the same manner as when synthesizing a dendrimer anthracene derivative, alternately reacting it with ethylenediamine and methyl acrylate. 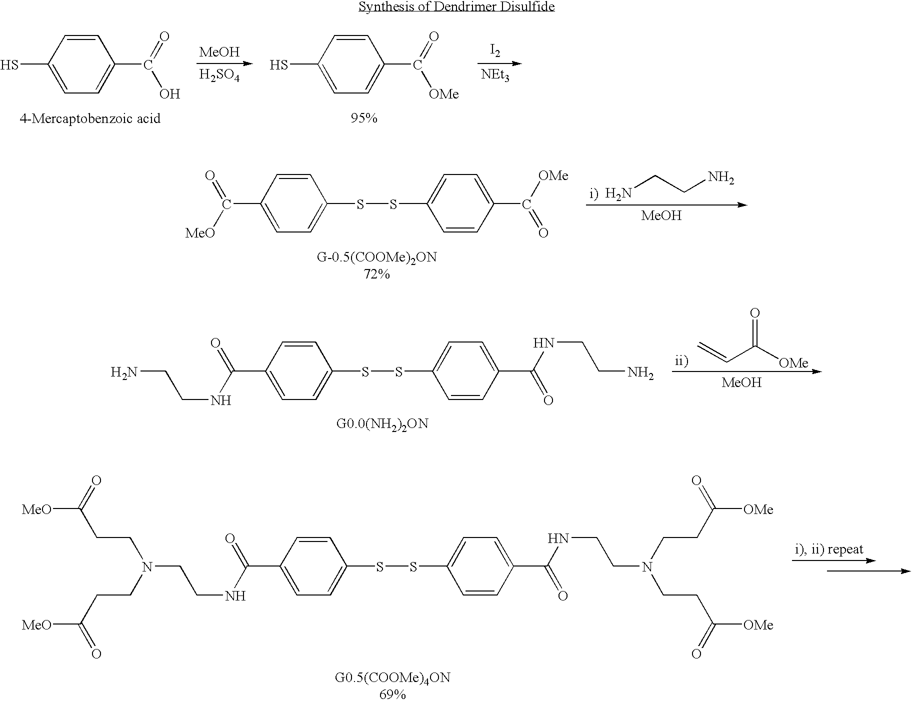 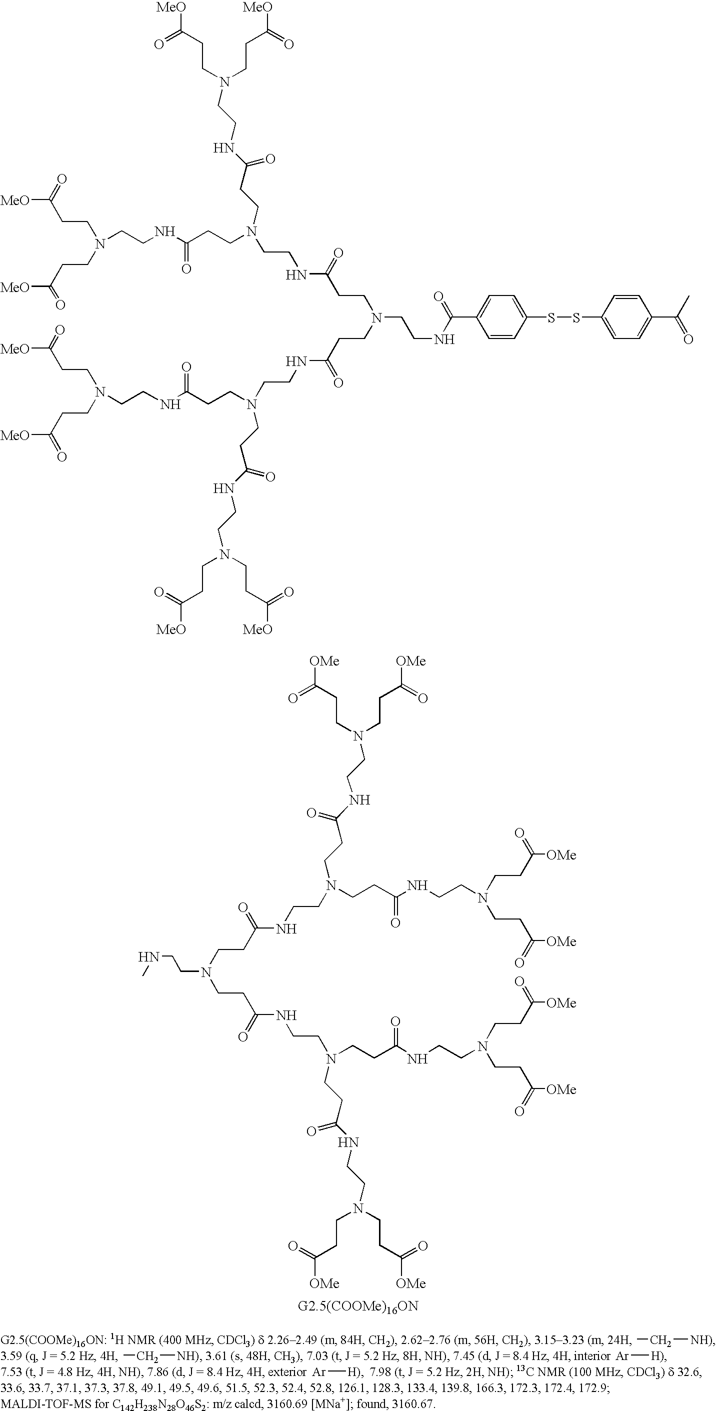 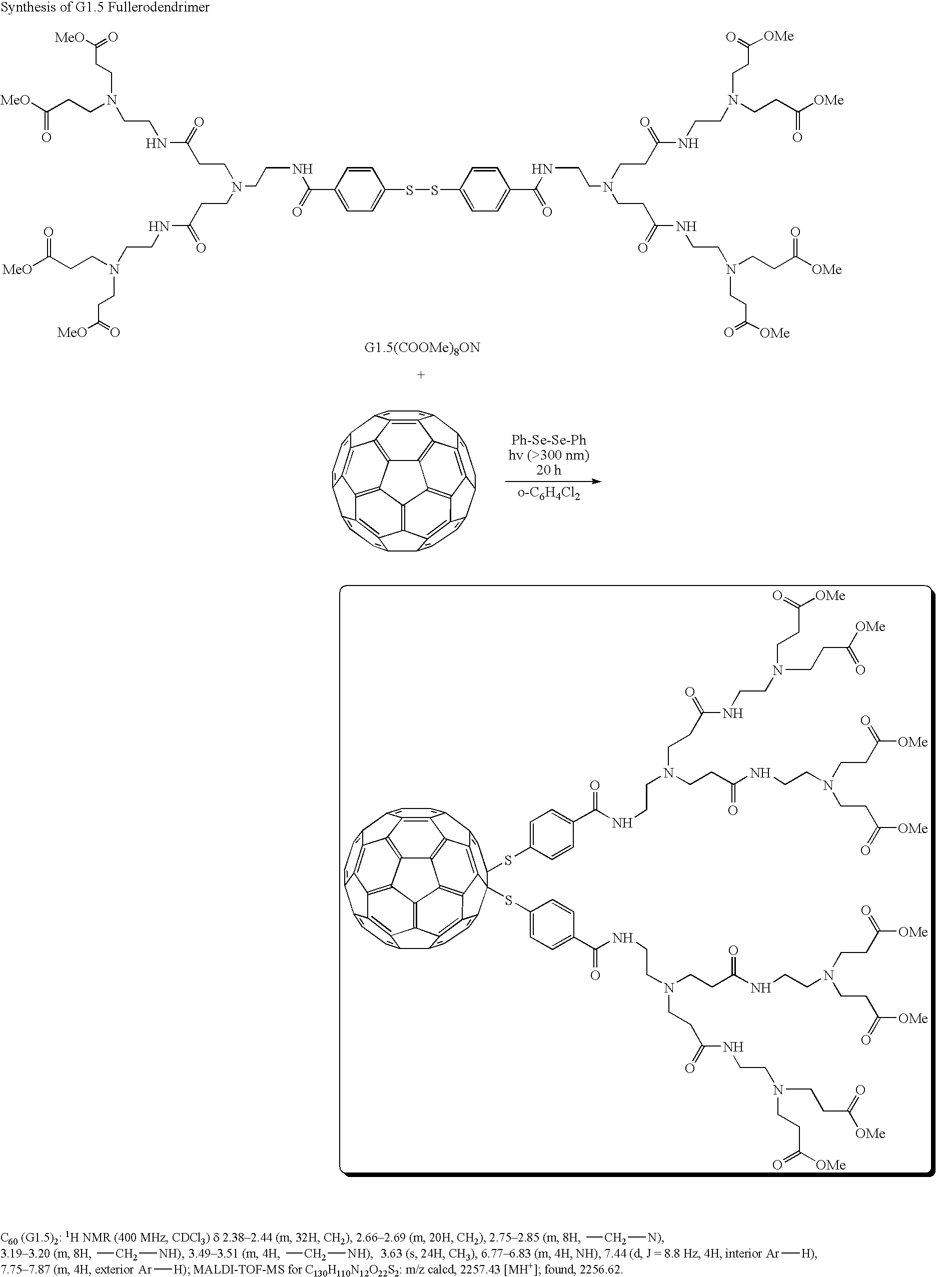 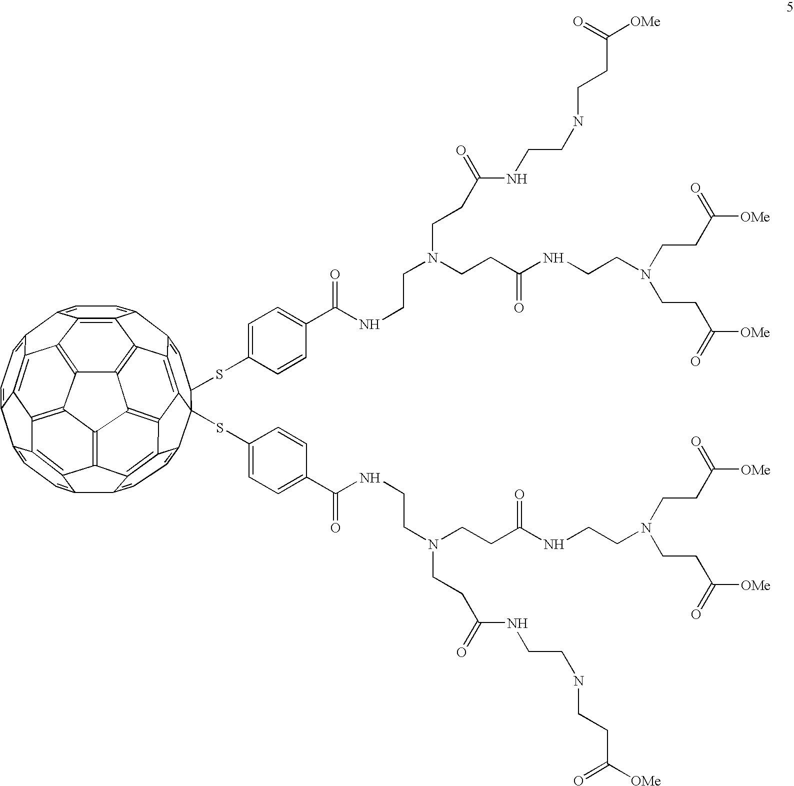
[Forming a Thin Film]

Fullerodendrimers are made to exhibit extremely high solubility in all solvents and polymeric substances by changing the terminal function group. Accordingly, it is easy to form thin films with fullerodendrimers. Until now, there has been inadequate applicational research into fullerenes irrespective of their high functionality. The main reason for this has been the difficulty of processing due to low solubility.

Thin films containing fullerodendrimers can be formed by dissolving a fullerodendrimer in various solvents and then employing spin coating, for example. Optimization of fullerodendrimer molecular design and film forming conditions permits the obtaining of more uniform films. Since it is possible to form thin films by spin coating, a considerable advantage is afforded in practical terms.

[The Preparation of Composite Films with Fullerodendrimers]

Fullerodendrimers have high affinity for various macromolecules, yielding optimal molecular designs in the creation of composite films (films containing fullerodendrimers and polymers). Improvement in optical carrier generation efficiency and the like by doping fullerene into polyvinylcarbazol has been reported. However, the solubility of C₆₀ in such polymers, at less than or equal to several weight %, is extremely low, making it difficult to prepare composite films having practical characteristics. By contrast, the fullerodendrimer employed in the present invention can be molecularly designed to have affinity for any and all polymers by changing the terminal functional group. Accordingly, it is possible to mix polymers having photoconductivity and fullerodendrimers to prepare new films.

Coating materials may also be prepared with the above-described fullerodendrimers and a binder. The binder may be either an organic or inorganic binder. Examples of inorganic binders are alkyl silicates; silicon halides; products obtained by hydrolyzing hydrolyzable silicon compounds, such as partially hydrolyzed products of the above; silicon compounds such as silica, colloidal silica, water glass, and organopolysiloxane; polycondensation products of organic polysiloxane compounds; and alumina compounds. Examples of organic binders are fluoropolymers, silicon polymers, acrylic resin, epoxy resin, polyester resin, melamine resin, urethane resin, and alkyd resin, as well as other known electroconductive resins and photosetting resins. In the present invention, these binders may be employed singly or in combinations of two or more.

The present invention relates to products having a coating of the above-described present invention on at least a portion of the surface of a substrate. Examples of substrates are metal, resin, ceramic, and glass. The product having a film containing the fullerodendrimer of the present invention can be employed to impart the various functions of fullerenes and fullerodendrimers to the substrate.

Examples of coating methods are application by the usual methods such as impregnation, deep coating, spin coating, blade coating, roller coating, wire bar coating, reverse roll coating, brush coating, and sponge coating, as well as spray application by the usual spray coating methods. Following this coating or spray coating, if the binder employed is resistant to high temperature, it is possible to heat the fullerene portion of the fullerodendrimer that has integrated with the binder, thereby eliminating or reducing it. There are cases in which heating is desirably conducted to a temperature of greater than or equal to 500° C. in a reducing atmosphere.

It is anticipated that the simple formation of thin films by spin coating will have a major impact on the practical development of organic semiconductor devices. For example, when the fullerene-containing film obtained is employed as an n-type amorphous organic semiconductor thin film and combined with a p-type organic semiconductor thin film to obtain a laminated film, it is thought to behave in the same manner as the p/n junctions seen in inorganic semiconductors and permit application to diode characteristics, photoelectric conversion (solar panels), and the like. Fullerene functions as a singlet oxygen sensitizer, and has an environmental purifying effect based on the photodecomposition of harmful substances in the air, including NOx and SOx compounds. Accordingly, it can be expected to function as a photocatalyst in the fullerene-containing thin film that is prepared, particularly with regard to environmental purification effects.

The present invention covers products comprising a substrate provided with a fullerene thin film. Such products can be manufactured by providing a fullerodendrimer-containing thin film on a substrate and heating the product in a non-oxidizing atmosphere to decompose the dendrimer constituting the fullerodendrimer.

### Embodiments

The present invention is described in greater detail below through embodiments.

All solvents and reagents were purchased from Aldrich, Kanto Kagaku K.K., Tokyo Kasei Kogyo K.K., and Wako Junyaku Kogyo K.K. The nuclear magnetic resonance (NMR) spectra were measured with a JEOL PMX60 (60 MHz) and Bruker AVANCE400 spectrometer (400 MHz). TMS was employed as the internal standard substance. Fractional high-performance liquid crystal chromatography (HPLC) was conducted with a Japan Analytical Co. model LC-918V. The columns employed were JAIGEL 1H, 2.5H (eluent: CHCl₃); 2.5H, 3H (eluent: CHCl₃), and JAIGEL GS-320 (eluent: MeOH). The MALDI-TOF-MS employed was a PerSeptive Biosystems Voyager Elite. The ultimate analyzer employed was a Perkin-Elmer 2400CHN.

1. Synthesis of Polyamidoaminedendrons having Anthracene Skeletons

### EXAMPLE 1-1

### Synthesis of 2-anthracenemethyl Carboxylate

2-Anthracenecarboxylic acid (1) (0.50 g, 225 mmol) was mixed with methanol (75 mL, 2.48×10³ mmol) and chloroform (60 mL). Ultrasound was applied for dissolution, after which sulfuric acid (7 mL) was added and the mixture was stirred with heating for 19 hours at 45° C. When the reaction had stopped, water (120 mL) was added, the mixture was transferred to a separating funnel, and separation was conducted. The organic layer was then washed twice with an aqueous solution of sodium bicarbonate, dehydrated with magnesium sulfate anhydride, and filtered with creased filter paper. The solvent was removed from the filtrate with an evaporator. The solid obtained was vacuum dried, yielding 2-methyl carboxylate (0.51 g, 2.20 mmol, 98% yield).

2-Methyl carboxylate (2):

¹H NMR (CDCl₃) δ 3.99 (s, 1H), 7.48-7.52 (m, 2H), 7.98-8.03 (m, 4H), 8.41 (s, 1H), 8.53 (s, 1H), 8.78 (s, 1H).

### EXAMPLE 1-2

### Synthesis of Generation 0.0 Polyamidoaminedendrons (G0.0 (NH₂)

To an eggplant-shaped flask was charged ethylenediamine (59.3 mL, 0.88 mmol). A methanol solution (59.3 mL) of 2-anthracenemethyl carboxylate (2) (0.519 g, 2.20 mmol) was added dropwise in small amounts with a Pasteur with ice cooling. With the completion of the dropwise addition, a calcium chloride tube was applied and stirring was conducted with heating for 20 hours at 45° C. A trap was applied and the reaction solution was vacuum dried with heating. A trace amount of methanol and an excess of diethylether were added, ultrasound was applied for about 20 min to conduct reprecipitation, and suction filtration was conducted with a Kiriyama funnel. The solid obtained was then vacuum dried, yielding generation 0.0 polyamidoaminedendron (G0.0 (NH₂)) (0.576 g, 99% yield).

### EXAMPLE 1-3

### Synthesis of Generation 0.5 Polyamidoaminedendron (G0.5 (COOMe)₂OFF)

Generation 0.0 polyamidoaminedendron (G0.0 (NH₂)) (0.576 g, 2.18 mmol) was dissolved in MeOH (80 mL), methyl acrylate (1.96 mL, 21.8 mmol) was added, a calcium chloride tube was applied, and stirring was conducted with heating for three days at 45° C. An evaporator was employed to remove the solvent from the reaction solution at less than or equal to 50° C. and drying was conducted under vacuum. Refinement by column chromatography (silica gel, eluent: chloroform) yielded generation 0.5 polyamidoaminedendron (G0.5 (COOMe)₂OFF) (1.213 g, 2.78 mmol, 85% yield). Generation 0.5 polyamidoaminedendron (G0.5 (COOMe)₂OFF):

¹H NMR CDCl₃) δ 2.48 (t, J=6.4 Hz, 2H), 2.70 (t, J=4.8 Hz, 2H), 2.80 (t, J=6.4 Hz, 4H), 3.54 (s, 6H), 3.62-3.66 (q, J=5.6 Hz, 2H), 7.35 (brs, 1H), 7.48-7.50 (m, 2H), 7.91-8.05 (m, 4H), 8.43 (s, 1H), 8.63 (s, 1H); Anal. Calcd. For C₂₅H₂₈N₂O₅: C, 68.79; H, 6.47; N, 6.42. Found: C, 68.45; H, 6.58; N, 6.34.

### EXAMPLE 1-4

### Synthesis of Generation 1.0 Polyamidoaminedendron (G1.0 (NH₂)₂)

To an eggplant-shaped flask was charged ethylenediamine (61.5 mL, 0.92 mmol). A methanol solution (123 mL) of generation 0.5 dendron (G0.5 (COOMe)₂OFF) (1.00 g, 2.3 mmol) was added dropwise in small amounts with a tap funnel with ice cooling. With the completion of the dropwise addition, a calcium chloride tube was applied and stirring was conducted for 21 hours at room temperature. A trap was applied and the reaction solution was vacuum dried with heating. A trace amount of methanol and an excess of diethylether were added, ultrasound was applied for about 20 min to conduct reprecipitation, and the supernatant was gradually removed. The solid obtained was then vacuum dried, yielding generation 1.0 polyamidoaminedendron (G1.0 (NH₂)₂)(0.833 g, 1.7 mmol, 74% yield).

### EXAMPLE 1-5

### Synthesis of Generation 1.5 Polyamidoaminedendron (G1.5(COOMe)₄)

Generation 1.0 polyamidoaminedendron (G1.0 (NH₂)₂) (0.833 g, 1.7 mmol) was dissolved in MeOH (120 mL), methyl acrylate (3 mL, 34 mmol) was added, a calcium chloride tube was applied, and stirring was conducted with heating for 43 hours at 45° C. An evaporator was employed to remove the solvent from the reaction solution at less than or equal to 50° C. and drying was conducted under vacuum. Refinement by column chromatography (silica gel, eluent: chloroform:methanol=40:1) yielded generation 1.5 polyamidoaminedendron (G1.5 (COOMe)₄OFF) (1.213 g, 2.78 mmol, 85% yield).

Generation 1.5 polyamidoaminedendron (G1.5 (COOMe)₄OFF):

¹HNMR (CDCl₃) δ 2.43 (t, J=6.8 Hzm 8H), 2.49 (t, J=5.6 Hz, 4H), 2.54 (t, J=5.6 Hz, 4H), 2.72 (t, J=6.8 Hz, 8H), 2.86 (t, J=5.6 Hz, 2H), 2.98 (t, J=5-6 Hz, 4H), 3.30-3.35 (q, J=5.6 Hz, 4H), 3.73 (s, 12H), 3.79-3.83 (q, J=5.6 Hz, 2H), 6.98 (t, J=5.2 Hz, 2H), 7.59-7.62 (m, 2H), 7.49-8.04 (m, 5H), 8.42 (s, 1H), 8.55 (s, 1H), 8.72 (s, 1H); 13C NMR (CDCl₃) δ 32.4, 33.7, 36.9, 37.5, 48.9, 49.2, 51.4, 52.6, 123.4, 125.5, 125.8, 125.9, 127.8, 127.9, 128.0, 128.1, 128.4, 130.5, 131.2, 131.7, 131.9, 132.4, 167.1, 172.2, 172.8; MALDI-TOF-MS for C₄₃H₆₀N₆₀O₁₁: m/z calcd, 836.97 [MH⁺]; found, 837.83.

### EXAMPLE 1-6

### Synthesis of Generation 2.0 Polyamidoaminedendron (G2.0 (NH₂)₄

To an eggplant-shaped flask was charged ethylenediamine (16.6 mL, 0.25 mmol). A methanol solution (33.2 mL) of generation 1.5 dendron (G1.5 (COOMe)₄OFF) (0.213 g, 2.2 mmol) and MeOH (32 mL) was added dropwise in small amounts with a tap funnel with ice cooling. With the completion of the dropwise addition, a calcium chloride tube was applied and stirring was conducted for 21 hours at room temperature. A trap was applied and the reaction solution was vacuum dried with heating. A trace amount of methanol and an excess of diethylether were added, ultrasound was applied for about 20 min to conduct reprecipitation, and the supernatant was gradually removed. The solid obtained was then vacuum dried, yielding generation 2.0 polyamidoaminedendron (G2.0 (NH₂)₄)(0.213 g, 1.1 mmol, 50% yield).

### EXAMPLE 1-7

### Synthesis of Generation 2.5 Polyamidoaminedendron (G2.5 (COOMe)₈)

Generation 2.0 polyamidoaminedendron (G2.0 (NH₂)₄) (0.213 g, 0.22 mmol) was dissolved in MeOH (32 mL), methyl acrylate (0.8 mL, 9.0 mmol) was added, a calcium chloride tube was applied, and stirring was conducted with heating for 43 hours at 45° C. An evaporator was employed to remove the solvent from the reaction solution at less than or equal to 50° C. and drying was conducted under vacuum. Refinement by column chromatography (silica gel, eluent: chloroform:methanol=10:1) yielded generation 2.5 polyamidoaminedendron (G2.5 (COOMe)₈OFF) (0.180 g, 0.11 mmol, 50% yield).

Generation 2.5 polyamidoaminedendron (G2.5 (COOMe)₈OFF):

¹H NMR (CDCl₃) δ 2.29 (t, J=6.4 Hz, 8H), 2.37-2.41 (m, 16H), 2.45-2.51 (m, 16H), 2.70-2.76 (m, 26H), 2.86 (t, J=6.0 Hz, 4H), 3.23-3.25 (m, 12H), 3.63 (s, 24H), 3.67-3.69 (q, J=4.0 Hz, 2H), 7.01 (t, J=5.2 Hz, 4H), 7.45-7.49 (m, 2H), 7.62 (t, J=4.4 Hz, 2H), 7.99-8.01 (m, 4H), 8.17 (t, J=4.8 Hz, 1H), 8.42 (s, 1H), 8.56 (s, 1H), 8.73 (s, 1H); ¹³C NMR (CDCl₃) δ 32.1, 32.6, 33.8, 34.0, 37.1, 37.4, 37.8, 49.1, 49.2, 49.6, 51.5, 52.4, 52.8, 123.5, 125.6, 125.9, 126.0, 128.0, 128.1, 128.2, 128.3, 128.6, 130.7, 131.3, 131.9, 132.1, 132.5, 167.3, 172.3, 172.4, 173.0; MALDI-TOF-MS for C₇₉H₁₂₄N₁₄O₂₃, m/z calcd, 1636.90[MH⁺]; found, 1637.43.

### EXAMPLE 1-8

### Synthesis of Generation 1.0 Dendrimer (G1.0 (CO₂t-BBu)₃)

Generation 1.0 dendrimer (G1.0 (NH₂)₃) was reacted overnight with a dimethylformamide solution (50 mL) of Behera's amine (H₂NC(CH₂CH₂CO₂t-Bu)₃) (187 mg, 0.45 mmol) and 2-anthracenecarboxylic acid (100 mg, 0.45 mmol) in the presence of dicyclohexylcarbodiimide, yielding generation 1.0 dendrimer (G1.0 (CO₂t-Bu)₃) (231 mg) in the form of white crystals. The terminal carboxylic ester groups were hydrolyzed to convert them to carboxylic acid, and then reacted with ethylenediamine. The (G1.0 (NH₂)₃) obtained was employed in the following reaction without refinement. Spectral data of generation 1.0 dendrimer (G1.0(CO₂t-Bu)₃):

¹H NMR (400 MHz, CDCl₃) δ 1.44 (s, 27H), 2.18-2.22 (m, 6H), 2.35-2.39 (m, 6H), 7.08 (s, 1H), 7.48-7.53 (m, 2H), 7.83 (d, 1H), 8.00-8.04 (dd, 3H), 8.43 (s, 1H), 8.50 (s, 1H), 8.51 (s, 1H); ¹³C NMR (100 MHz, CDCl₃)δ (28.1, 30.0, 30.3, 58.0, 80.8, 123.0, 125.7, 126.18, 126.22, 127.98, 128.03, 128.2, 128.6, 130.6, 1.31.7, 132.11, 132.13, 132.7, 166.7, 173.1.

2. Bond Generation by the Diels-Alder Reaction of Polyamidoaminedendron having an Anthracene Skeleton and C₆₀ Fullerene

### EXAMPLE 2.1

### Synthesis of Generation 0.5 Fullerodendrimer

C₆₀ (50 mg, 6.93×10⁻² mmol) was added to o-C₆H₄Cl₂ (3.9 mL) in a threaded-neck test tube and fully dissolved by applying ultrasound. To this was added and dissolved generation 0.5 polyamidoaminedendron (G0.5(COOMe)₂OFF) (0.061 g, 0.14 mmol), the test tube was back-filled with N₂, the solution was sealed in the test tube, and stirring was conducted with heating for four days at 45° C. The reaction solution was refined by column chromatography (silica gel, eluent: CHCl₃), yielding generation 0.5 fullerodendrimer ([G0.5-C₆₀]adduct) (56 mg, 4.84×10⁻² mmol, 70% yield).

Generation 0.5 fullerodendrimer ([G0.5-C₆₀]adduct):

¹H NMR(CDCl₃) δ 2.46 (t, J=6.0 Hz, 4H), 2.69 (t, J=6.0 Hz, 2H), 2.77-2.81 (m, 4H), 3.55 (s, 6H), 3.60-3.65 (m, 2H), 5.84 (s, 1H), 5.88 (s, 1H), 7.29 (t, J=8.0 Hz, 1H), 7.47-7.49 (m, 2H), 7.75-7.76 (m, 2H), 7.82 (d, J=8.0 Hz, 1H), 8.02 (t, J=4.0 Hz, 1H), 8.38 (s, 1H), ¹³C NMR (CDCl₃) δ 29.7, 32.7, 37.4, 48.9, 51.6, 52.8, 58.2, 58.3, 72.3, 72.3, 125.0, 125.7, 125.9, 126.0, 126.3, 127.5, 127.5, 133.6, 136.9, 136.9, 137.0, 139.9, 139.9, 141.1, 141.4, 141.6, 141.6, 142.0, 142.0, 142.0, 142.2, 142.2, 142.3, 142.5, 143.0, 143.0, 144.5, 144.6, 144.6, 144.9, 145.2, 145.2, 145.3, 145.3, 145.3, 145.4, 145.6, 146.1, 146.1, 146.2, 146.4, 146.4, 147.5, 147.5, 155.2, 155.3, 155.3, 167.0, 173.0; MALDI-TOF-MS for C₈₅H₂₈N₂O₅: m/z calcd 1157.14, [M]; found, 1156.54.

### EXAMPLE 2-2

### Synthesis of Generation 1.5 Fullerodendrimer

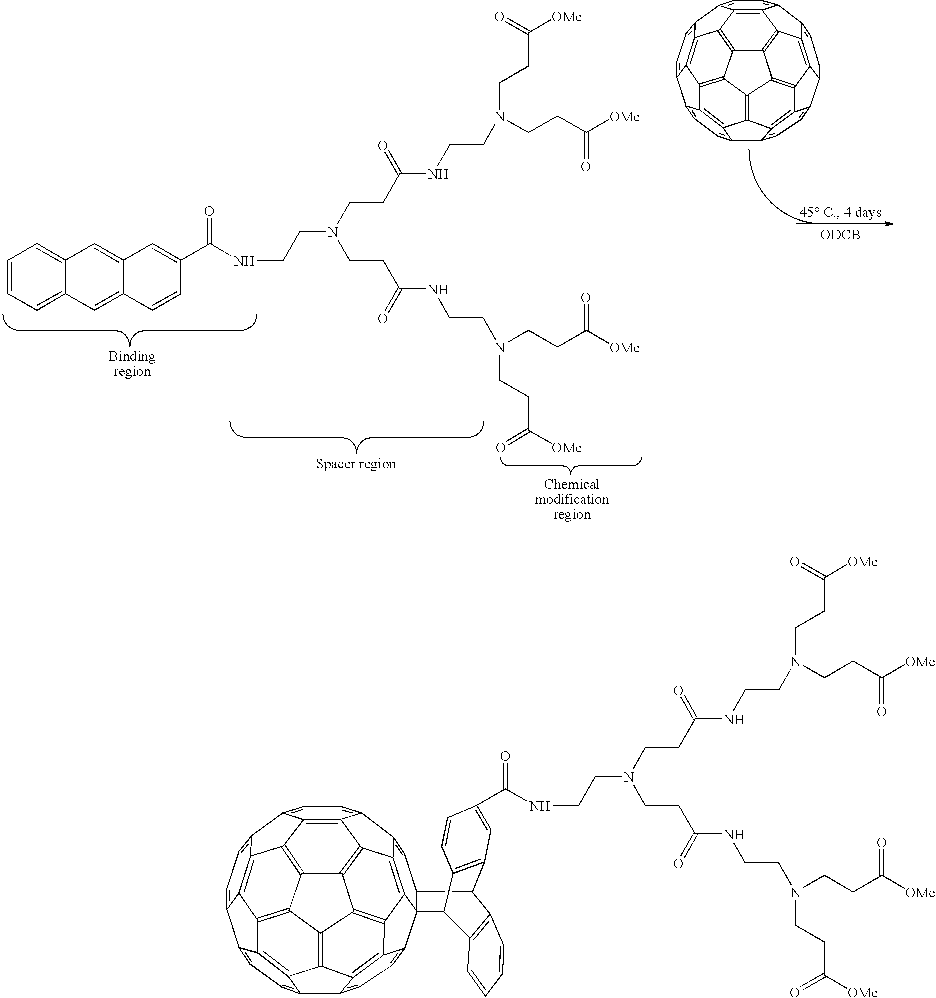 (Experiment)

To an o-C₆H₄Cl₂ solution (3.4 mL) of C₆₀ (44 mg, 6.15×10⁻² mmol) was added generation 1.5 polyamidoaminedendron (G1.5 (COOMe)₄OFF) (0.103 g, 0.12 mmol) and the mixture was stirred with heating for four days at 45° C. in an N₂ atmosphere. The reaction solution was then refined by column chromatography (silica gel, eluent: CHCl₃:MeOH=40:1), yielding generation 1.5 fullerodendrimer ([G1.5-C₆₀]adduct) (65 mg, 4.17×10⁻² mmol, 70% yield) in the form of an oily, brown substance.

(Spectral Data)

¹H NMR (CDCl₃) δ (2.24-2.35 (m, 16H), 2.55 (t, J=6 Hz, 8H), 2.63-2.82 (m, 6H), 3.12 (q, J=5 Hz, 4H), 3.54-3.65 (m, 14H), 5.78 (s, 1H), 5.82 (s, 1H), 6.79 (t, J=4 Hz, 2H), 7.38-7.43 (m, 2H), 7.67-7.71 (m, 2H), 7.74 (d, J=8 Hz, 1H), 7.79-7.93 (m, 1H), 8.04 (d, J=8 Hz, 1H), 8.39 (s, 1H); ¹³C NMR (CDCl₃) (32.7, 33.8, 37.1, 37.7, 49.1, 51.6, 52.4, 52.8, 52.8, 71.5, 71.5, 125.2, 125.5, 125.7, 125.9, 126.0, 126.2, 126.4, 126.6, 127.2, 127.4, 127.5, 128.3, 133.5, 133.7, 136.1, 139.4, 141.1, 141.3, 141.4, 141.7, 141.8, 142.0, 142.1, 142.3, 142.4, 142.4, 142.7, 142.8, 142.8, 143.5, 143.6, 143.8, 143.9, 144.0, 144.0, 144.1, 144.7, 144.8, 144.9, 145.1, 145.2, 145.3, 145.4, 145.5, 145.7, 145.7, 145.8, 146.1, 146.2, 147.1, 147.1, 147.2, 147.8, 147.8, 148.1, 148.2, 148.3, 148.5, 148.6, 149.1, 155.0; MALDI-TOF-MS for C₁₀₃H₆₀N₆O₁₁: m/z calcd 1557.61, [M⁻]; found, 1556.84.

### EXAMPLE 2-3

### Synthesis of Generation 2.5 Fullerodendrimer

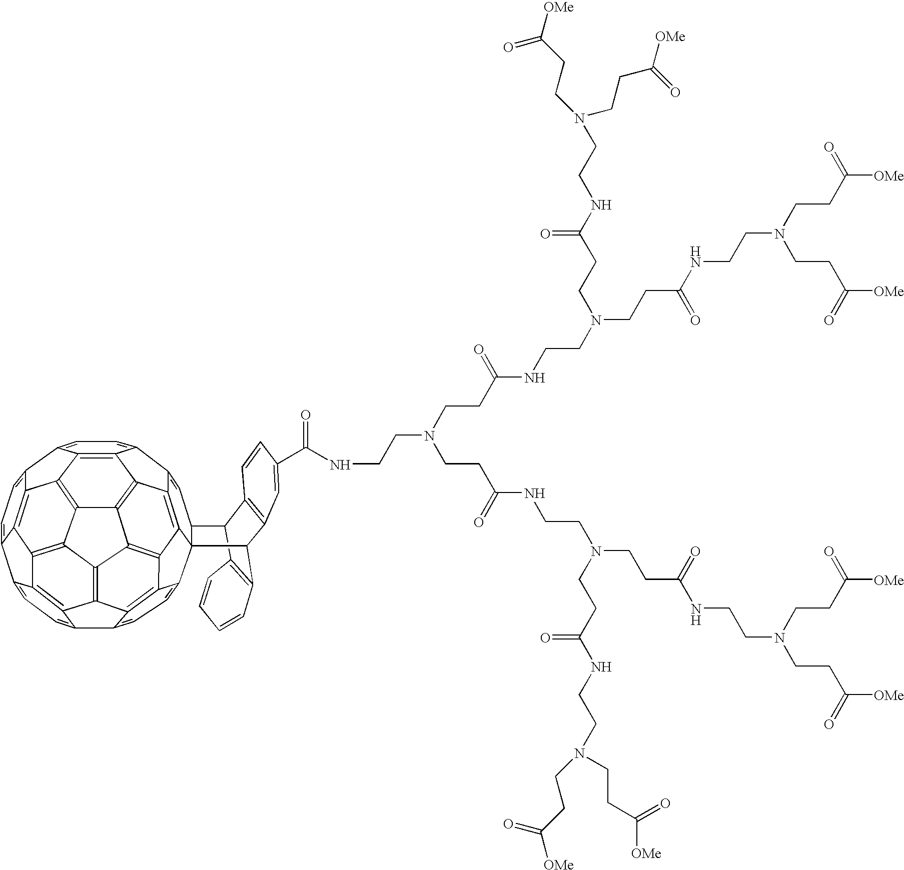 (Experiment)

To an o-C₆H₄Cl₂ solution (2.4 mL) of C₆₀ (30 mg, 3.84×10⁻² mmol) was added generation 2.5 polyamidoaminedendron (G2.5 (COOMe)₈OFF) (0.126 g, 7.67×10⁻² mmol) and the mixture was stirred with heating for four days at 45° C. in an N₂ atmosphere. The reaction solution was then refined by column chromatography (silica gel, eluent: CHCl₃:MeOH=10:1), yielding generation 2.5 fullerodendrimer ([G2.5-C₆₀]adduct) (32 mg, 1.36×10⁻² mmol, 40% yield) in the form of an oily, brown substance.

(Spectral Data)

¹H NMR (CDCl₃) δ 2.25 (t, J=6 Hz, 8H), 2.32-2.36 (m, 27H), 2.46 (t, J=6 Hz, 10H), 2.60-2.68 (m, 28H), 2.72-2.79 (m, 6H), 3.12-3.21 (m, 12H), 3.59 (s, 24H), 5.79 (s, 1H), 5.83 (s, 1H), 6.95-6.97 (m, 2H), 7.38-7.43 (m, 2H), 7.69-7.72 (m, 2H), 7.75-7.77 (m, 1H), 8.04-8.06 (m, 2H), 8.40 (s, 1H); ¹³C NMR (CDCl₃) δ 14.1, 22.6, 29.3, 30.1, 30.3, 31.9, 32.6, 37.2, 49.2, 50.0, 51.7, 52.8, 58.0, 58.1, 72.3, 72.4, 125.5, 125.5, 125.9, 126.0, 126.1, 126.7, 127.5, 127.5, 129.6, 129.7, 130.0, 133.2, 136.8, 136.9, 136.9, 137.0, 139.8, 139.9, 141.3, 141.3, 141.6, 141.6, 141.7, 142.0, 142.0, 142.0, 142.2, 142.2, 142.3, 142.3, 142.5, 142.9, 142.9, 143.1, 143.1, 144.6, 144.6, 144.6, 144.7, 145.1, 145.2, 145.3, 145.3, 145.4, 145.4, 146.1, 146.2, 146.4, 146.4, 147.5, 147.5, 147.6, 147.6, 155.2, 155.3, 167.5, 170.8, 172.3, 173.0; MALDI-TOF-MS for C₁₃₉H₁₂₄N₁₄O₂₃: m/z calcd 2358.55, [M⁻]; found, 2356.73.

### EXAMPLE 2-4

### Synthesis of C₆₀-Generation 0.5 Polyamidoaminedendron-Generation 1.0 Terminal Oligoethyleneoxidedendron Adduct (C₆₀-G0.5(COOMe)₂-G1.0 (Oligoethyleneoxide)₂)

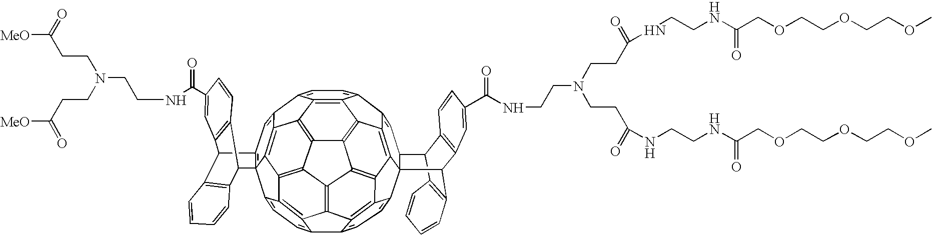 (Experiment)

A chloroform solution (1.5 mL) of generation 1.0 terminal oligoethyleneoxidedendron (G1.0 (oligoethyleneoxide)₂) (0.042 g, 0.0520 mmol) and C₆₀-generation 0.5 polyamidoaminedendron monoadduct (C₆₀-G0.5(COOMe)₂) (0.040 g, 0.0346 mmol) was stirred with heating for one week at 45° C. under a nitrogen atmosphere and the reaction solution was refined by fractional HPLC, yielding C₆₀-generation 0.5 polyamidoaminedendron-generation 1.0 terminal oligoethyleneoxidedendron adduct (C₆₀-G0.5(COOMe)₂-G1.0(oligoethyleneoxide)₂) (0.028 g, 41% yield) in the form of an oily, brown substance.

(Spectral Data)

¹H-NMR (CDCl₃) δ 1.77-2.14 (m, 8H), 2.33-2.55 (m, 4H), 2.57-2.92 (m, 8H), 3.07-3.33 (m, 16H), 3.34-3.70 (m, 26H), 3.84-3.96 (m, 4H), 5.60-6.13 (m, 4H), 7.21-8.61 (m, 16H).

### EXAMPLE 2-5

### Synthesis of C₆₀-Generation 0.5 Polyamidoaminedendron-Generation 2.0 Terminal Oligoethyleneoxidedendron Adduct (C₆₀-G0.5(COOMe)₂-G2.0 (Oligoethyleneoxide)₄) Adduct

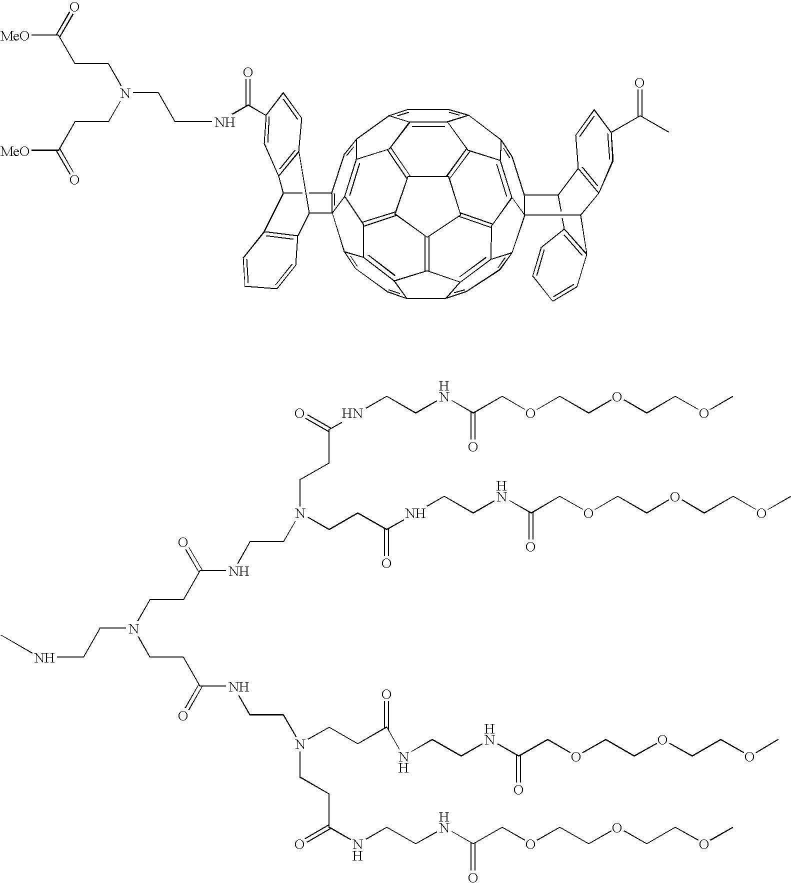 (Experiment)

A chloroform solution (1.5 mL) of generation 2.0 terminal oligoethyleneoxidedendron (G2.0 (oligoethyleneoxide)₄) (0.082 g, 0.0519 mmol) and C₆₀-generation 0.5 polyamidoaminedendron monoadduct (C₆₀-G0.5(COOMe)₂) (0.040 g, 0.0346 mmol) was stirred with heating for one week at 45° C. under a nitrogen atmosphere and the reaction solution was refined by fractional HPLC, yielding C₆₀-generation 0.5 polyamidoaminedendron-generation 2.0 terminal oligoethyleneoxidedendron adduct (C₆₀-G0.5(COOMe)₂-G2.0(oligoethyleneoxide)₄) adduct (0.024 g, 25% yield) in the form of an oily, brown substance.

(Spectral Data)

¹H-NMR (CDCl₃) δ 2.14-2.32 (m, 8H), 2.33-2.46 (m, 8H), 2.49-2.66 (m, 12H), 2.66-2.91 (m, 12H), 3.05-3.22 (m, 4H), 3.29-3.44 (m, 28H), 3.45-3.79 (m, 42H), 3.92-4.07 (m, 8H), 5.67-6.20 (m, 4H), 7.33-8.73 (m, 26H).

### EXAMPLE 2-6

### C₆₀-Generation 1.5 Polyamidoaminedendron-Generation 2.0 Terminal Oligoethyleneoxidedendron Adduct (C₆₀-G1.5(COOMe)₄-G2.0 (Oligoethyleneoxide)₄)

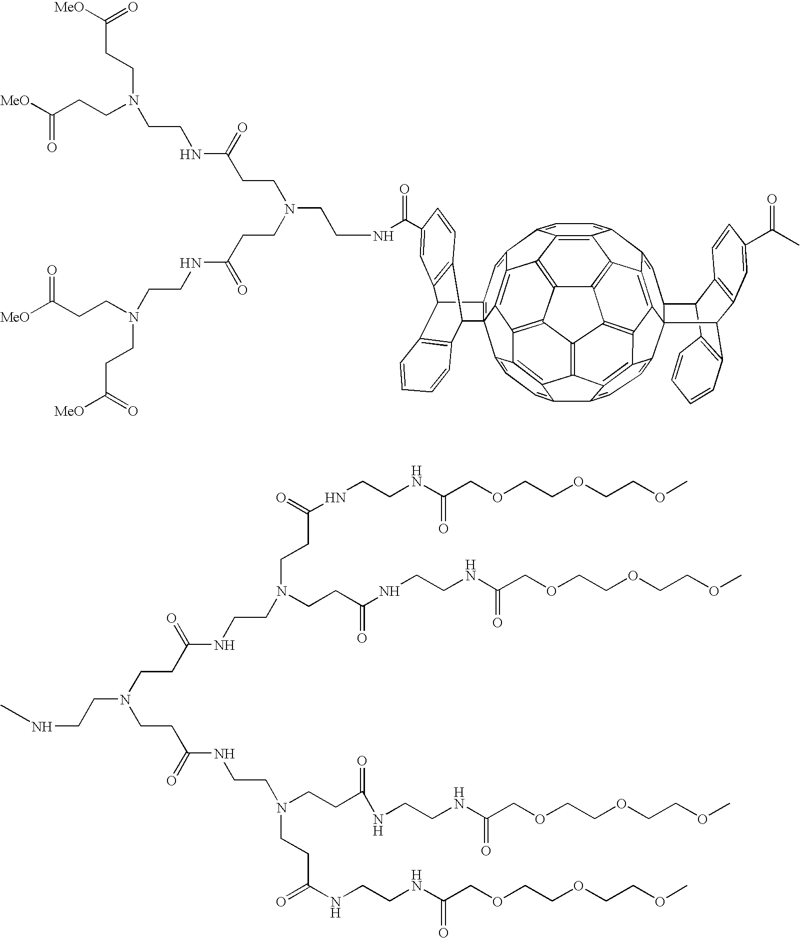 (Experiment)

A chloroform solution (0.75 mL) of generation 2.0 terminal oligoethyleneoxidedendron (G2.0 (oligoethyleneoxide)₄) (0.061 g, 0.0385 mmol) and C₆₀-generation 1.5 polyamidoaminedendron monoadduct (C₆₀-G1.5(COOMe)₄) (0.040 g, 0.0257 mmol) was stirred with heating for one week at 45° C. under a nitrogen atmosphere and the reaction solution was refined by fractional HPLC, yielding C₆₀-generation 1.5 polyamidoaminedendron-generation 2.0 terminal oligoethyleneoxidedendron adduct (C₆₀-G1.5(COOMe)₄-G2.0(oligoethyleneoxide)₄) (0.019 g, 23% yield) in the form of an oily, brown substance.

(Spectral Data)

¹H-NMR (CDCl₃) δ 2.16-2.46 (m, 32H), 2.48-2.65 (m, 16H), 2.65-2.91 (m, 12H), 3.05-3.26 (m, 8H), 3.29-3.44 (m, 28H), 3.52-3.78 (m, 48H), 3.95-4.01 (m, 8H), 5.68-6.19 (m, 4H), 6.77-6.92 (m, 2H), 7.28-8.69 (m, 26H).

### EXAMPLE 2-7

### Synthesis of Generation 1.0 Terminal Diphenylselenide Polyamidoaminedendron C₆₀ Adduct (G1.0 (diphenylselenide)₂-C₆₀)

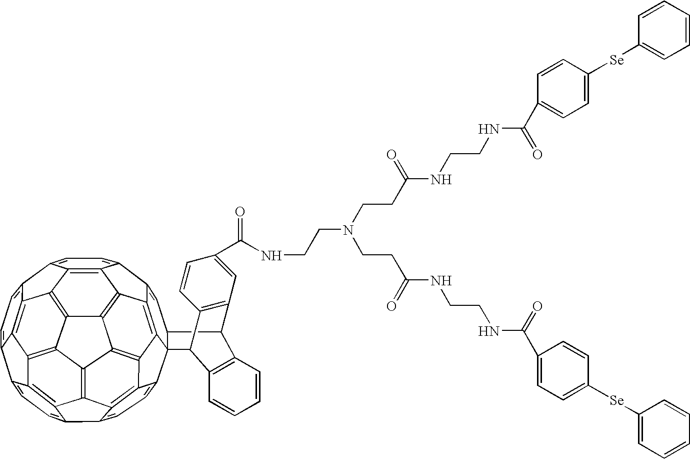 (Experiment)

Generation 1.0 terminal diphenylselenide polyamidoaminedendron (G1.0 (diphenylselenide)₂) (50 mg, 0.049 mol) and fullerene (C₆₀) (31 mg, 0.043 mol) were dissolved in a mixed solvent of o-dichlorobenzene/chloroform/methanol (3 mL, 1 mL, 0.5 mL) and reacted for seven days at 45° C. under a nitrogen atmosphere. Subsequently, the reaction solution was refined, yielding the targeted generation 1.0 terminal diphenylselenide polyamidoaminedendron C₆₀ adduct (G1.0 (diphenylselenide)₂-C₆₀) (22 mg, 0.013 mmol, 44% yield) in the form of an oily, brown substance.

(Spectra)

¹H NMR (CDCl₃) δ (2.29 (brs, 4H), 2.55-2.71 (m, 6H), 3.19-3.51 (m, 10H), 5.80 (s, 1H), 5.85 (s, 1H), 7.26-7.34 (m, 10H), 7.42-7.46 (m, 2H), 7.47-7.56 (m, 4H), 7.58-7.64 (m, 4H), 7.69-7.80 (m, 3H), 7.93 (brs, 1H), 8.05 (d, J=7.7 Hz, 1H), 8.38 (s, 1H); ¹³C NMR (CDCl₃) δ 33.8, 38.0, 39.1, 40.8, 50.2, 52.4, 58.1, 58.2, 72.3, 125.0, 125.9, 126.0, 126.4, 127.6, 127.8, 128.3, 128.4, 129.0, 129.7, 130.9, 132.0, 133.1, 134.6, 136.7, 136.8, 136.9, 137.6, 139.9, 141.0, 141.1, 141.2, 141.3, 141.5, 141.6, 141.7, 142.0, 142.1, 142.2, 142.3, 142.5, 142.9, 144.5, 144.6, 145.1, 145.2, 145.3, 145.4, 146.1, 146.2, 146.4, 147.5, 155.1, 155.2, 167.4, 167.8, 173.8; MALDI-TOF-MS for C₁₁₃H₅₂N₆O₅Se₂: m/z calcd, 1730.57 [M⁻]; found, 1730.17.

### EXAMPLE 2-8

### Synthesis of G1.0 Terminal Diphenylselenide Fullerodendron

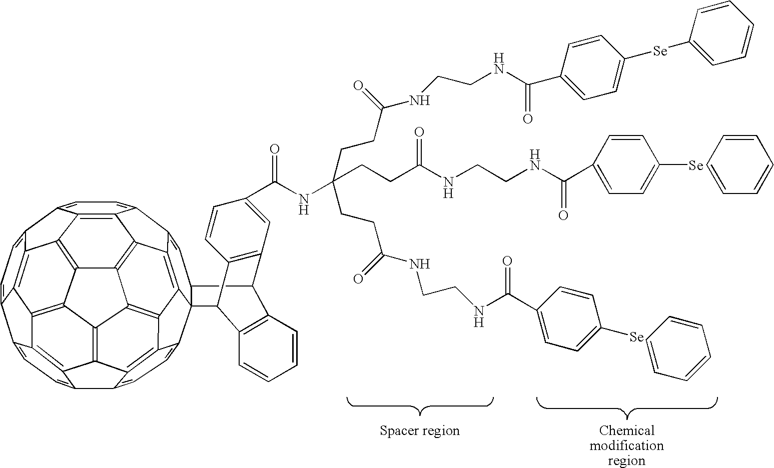 (Experiment)

Generation 1.0 dendrimer (G1.0 (diphenylselenide)₃) (48 mg, 0.0354 mmol) was dissolved in a mixed solution of o-dichlorobenzene (3 mL), chloroform (2 mL), and methanol (1 mL). C₆₀ (26 mg, 0.0361 mmol) was added and the mixture was stirred with heating for one week at 45° C. under a nitrogen atmosphere. The reaction solution was refined by silica gel chromatography (chloroform:methanol=20:1), yielding G1.0 terminal diphenylselenide fullerodendrimer (28 mg, 0.0135 mmol, 38% yield) in the form of an oil, black material.

(Spectra)

¹H NMR (CDCl₃) δ 2.04 (brs, 6H), 2.17 (brs, 6H), 3.34 (brs, 6H), 5.82 (s, 1H), 5.78 (s, 1H), 7.26-7.33 (m, 17H), 7.59 (d, J=8.0 Hz, 12H), 7.68-7.73 (m, 3H), 7.82 (d, J=7.7, 1H), 8.26 (s, 1H); MALDI-TOF-MASS for C₁₃₀H₆₇N₇O₇Se₃: m/z calcd, 2074.85 [M⁻]; found, 2074.13.

### EXAMPLE 2-9

### Synthesis of Terminal Methoxydilphenylselenide Fullerodendrimer

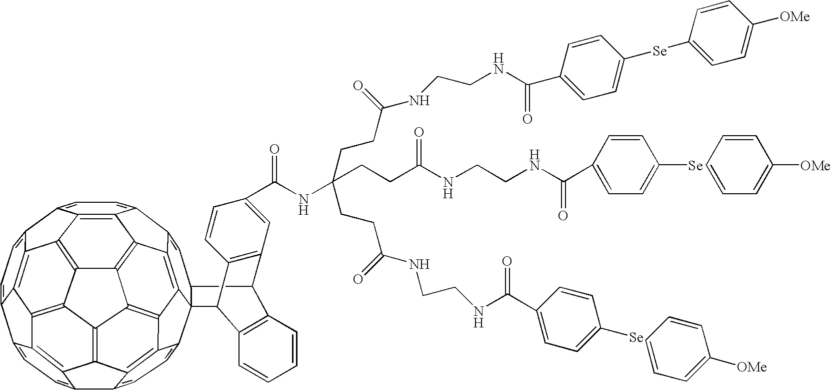 (Experiment)

To an o-dichlorobenzene solution (5 mL) of generation 1.0 dendrimer (G1.0 (methoxydiphenylselenide)₃) (4 mg, 0.00314 mmol) was added C₆₀ (5 mg, 0.00628 mmol) and the mixture was stirred with heating for one week in a 45° C. oil bath. Refinement was conducted by silica gel chromatography (chloroform:methanol=10:1), yielding terminal methoxydiphenylselenide fullerodendron (0.5 mg, 0.000251 mmol 8%) in the form of an oily, black substance.

(Spectra)

¹H NMR (CDCl₃) δ 2.36-2.41 (brs, 12H), 3.80 (s, 9H), 4.27-4.30 (m, 6H), 5.83 (s, 1H), 5.84 (s, 1H), 6.80-6.84 (m, 6H), 7.03-7.09 (m, 6H), 7.25-7.21 (m, 6H), 7.43-7.46 (m, 6H), 7.88 (s, 1H) 7.94 (brs, 1H), 7.96-8.04 (m, 3H), 8.28 (s, 1H).

### EXAMPLE 2-10

### Synthesis of Fulleropolyamidoaminedendron (mono[2-G2.0(2-fluorooctyl)ethyl Ester)₄]C₆₀ Adduct)

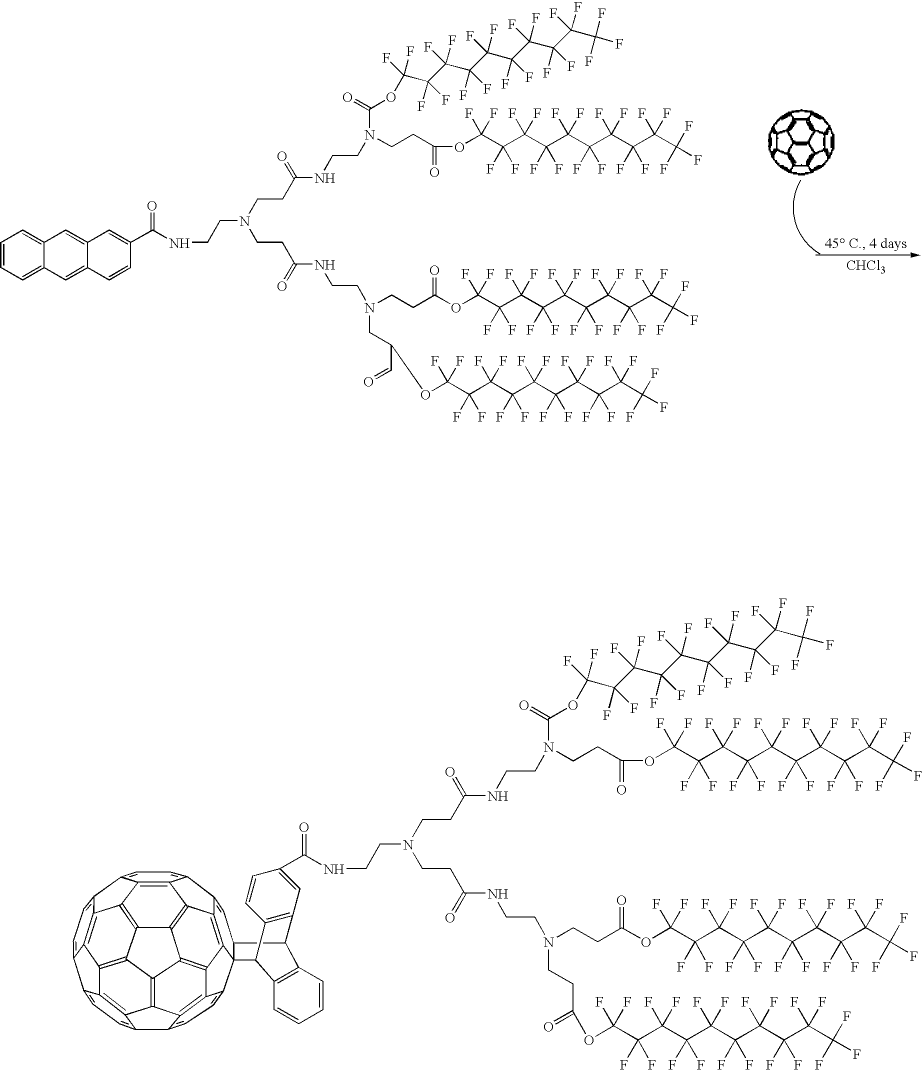 (Experiment)

To a mixed solution of chloroform (2.5 mL) and o-dichlorobenzene (5 mL) comprising generation 2.0 polyamidoaminedendron (2-G2.0(2-(fluorooctyl)ethyl ester)₄) (20 mg, 0.00779 mmol) was added C₆₀ (56 mg, 0.0777 mmol) and the mixture was stirred with heating for 12 days at 45° C. under a nitrogen atmosphere. The product was refined by HPLC (eluent: CHCl₃), yielding fulleropolyamidoaminedendron (mono[2-G2.0(2-(fluorooctyl)ethyl ester)₄] C₆₀ adduct) (63 mole %, 0.00397 mmol, 13 mg, 69% yield) in the form of an oily, brown substance.

(Spectra)

¹H NMR (CD₃Cl) δ 2.24-2.55 (m, 24H), 2.55-2.63 (m, 8H), 2.63-2.75 (m, 2H), 2.75-2.86 (m, 4H), 3.20-3.72 (m, 4H), 3.60-3.72 (m, 2H), 4.36 (t, J=6.4 Hz, 8H), 5.84 (s, 1H), 5.89 (s, 1H), 7.45-7.48 (m, 2H), 7.75-7.78 (m, 2H), 7.94 (brs, 1H), 8.12 (s, 1H), 8.46 (s, 1H);

¹⁹F NMR (CDCl₃) δ −126.7, −124.0, −123.3, −122.5, −122.5, −122.2, −114.2, −81.3; MALDI-TOF-MASS for C₁₃₉H₆₄F₆₈N₆O₁₁ m/z calcd, 3286.92 [M⁻]; found, 3285.88.

3. Synthesis of Polyamidoaminedendrimer Disulfides

### EXAMPLE 3-1

### Synthesis of 4,4′-dithiobismethyl Benzoate (3)

4-Mercaptobenzoic acid (1) was esterified with MeOH in the presence of concentrated sulfuric acid to synthesize 4-mercaptomethyl benzoate (2) (95% yield). The 4-mercaptomethyl benzoate (2) obtained was iodated in the presence of Net₃ to become the core of the dendrimer disulfide, yielding 4,4′-dithiobismethyl benzoate (3) (92% yield).

### EXAMPLE 3-2

### Synthesis of Polyamidoaminedendrimer Disulfide

Polyamidoaminedendrimer disulfide was synthesized by the divergent method, in which a dendrimer is synthesized from core to periphery. 4,4′-Dithiobismethyl benzoate (3) was reacted with ethylenediamine, and generation 0.0 polyamidoaminedendrimer disulfide (G0.0 (NH₂)₂ON) was synthesized (100% yield). The G0.0 (NH₂)₂ON was reacted with methyl acrylate to synthesize generation 0.5 polyamidoaminedendrimer disulfide (G0.5 (COOMe)₄ON (70% yield). The reactions with ethylenediamine and methyl acrylate were similarly repeated to obtain higher generations of generation 1.0 (G1.0 (NH₂)₄)ON, 100% yield), generation 1.5 (G1.5 (COOMe)₈ON, 92% yield), generation 2.0 ((G2.0 (NH₂)₈)ON, 92% yield), generation 2.5 (G2.5 (COOMe)₁₆ON, 68% yield), 3.0 generation ((G3.0 (NH₂)₁₆)ON, 100% yield), and generation 3.5 (G3.5 (COOMe)₃₂ON, 47% yield) polyamidoaminedendrimer disulfides. These polyamidoaminedendrimer disulfides corresponded to the ON state of the dendrimer structure.

The structures of the 0.5, 1.5, 2.5, and generation 3.5 dendrimers were determined by ¹H NMR, ¹³C NMR, and MALDI-TOF-MASS spectrometry. In MALDI-TOF-MASS spectrometry, in particular, 1.5, 2.5, and generation 3.5 dendrimer disulfide parent peaks were observed. Also of great interest, molecular ion peaks corresponding to half the molecular weights of the dendrimer disulfides were observed in each generation. This was attributed to thiyl radicals produced by cleavage of the disulfide bonds, with the thiyl radicals being presumed to be relatively stable. This suggests the possibility of utilizing the homolytic cleavage and rebonding of disulfide bonds in the control of reversible dendrimer structures. 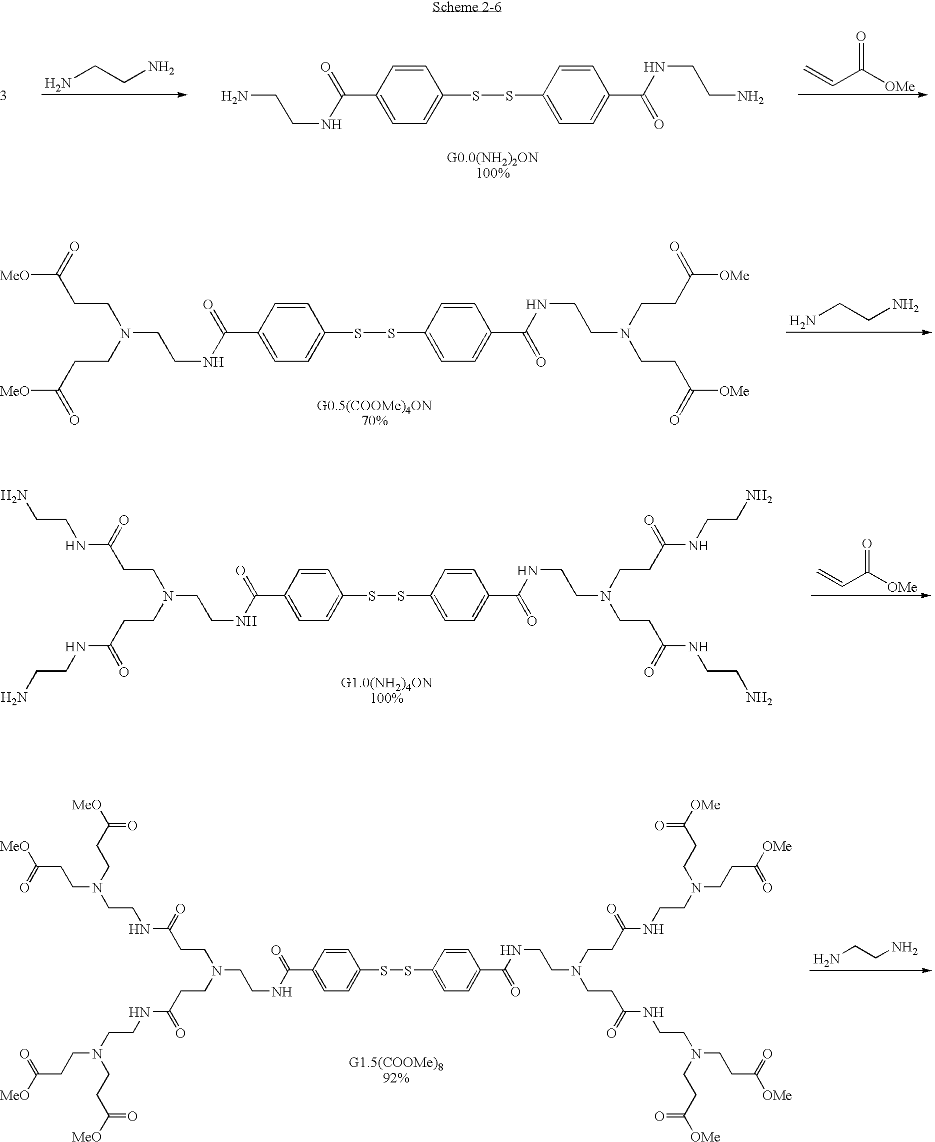 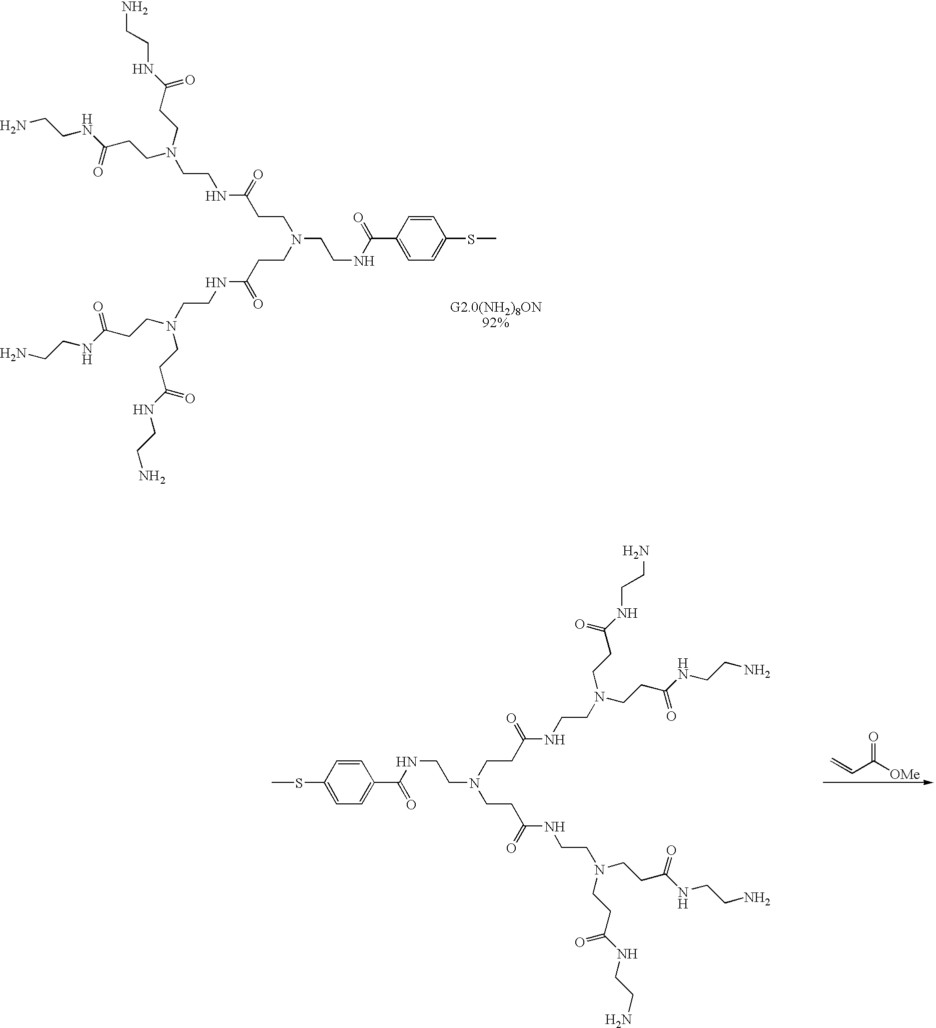 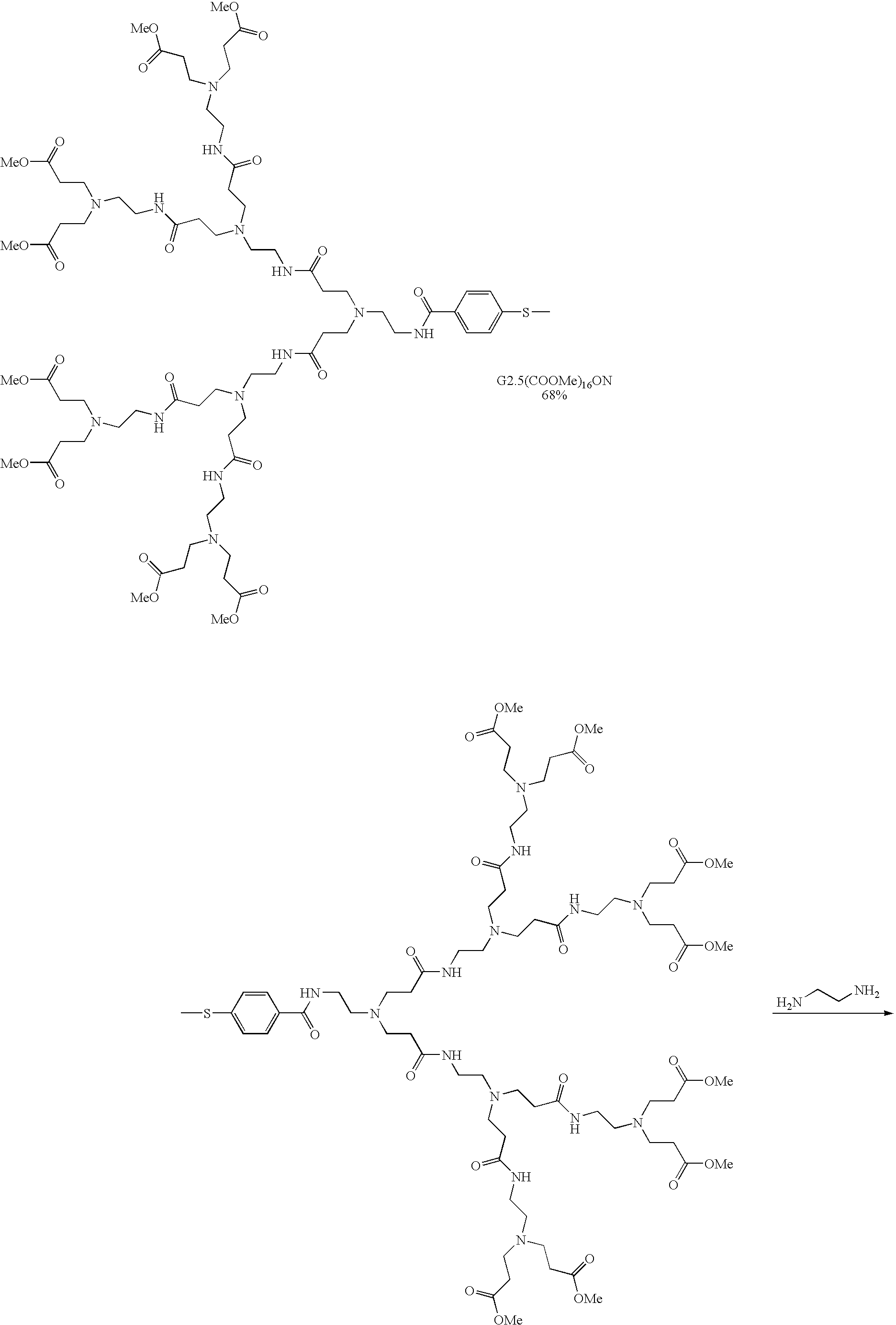 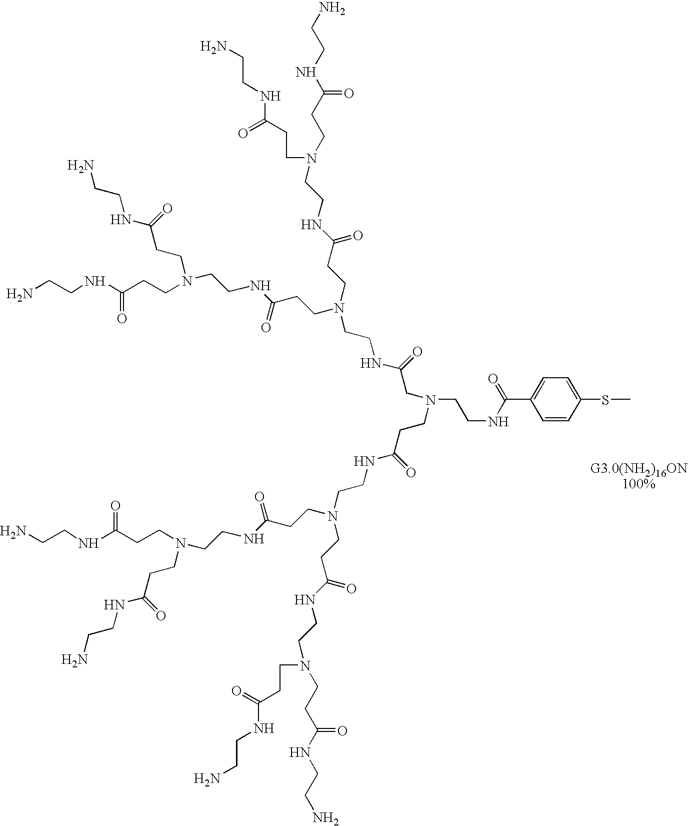 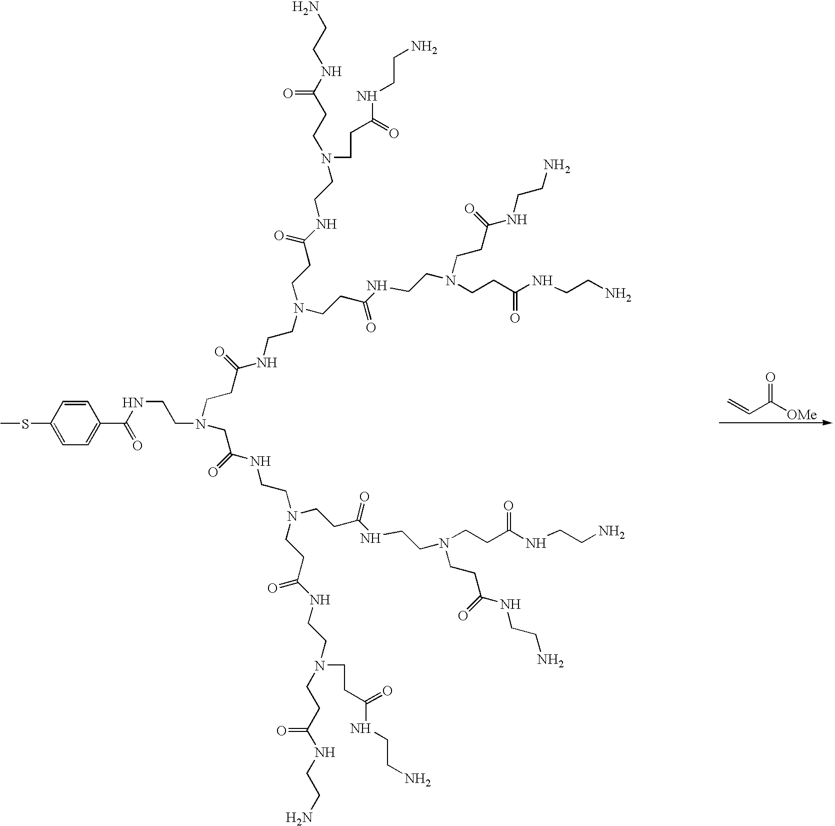 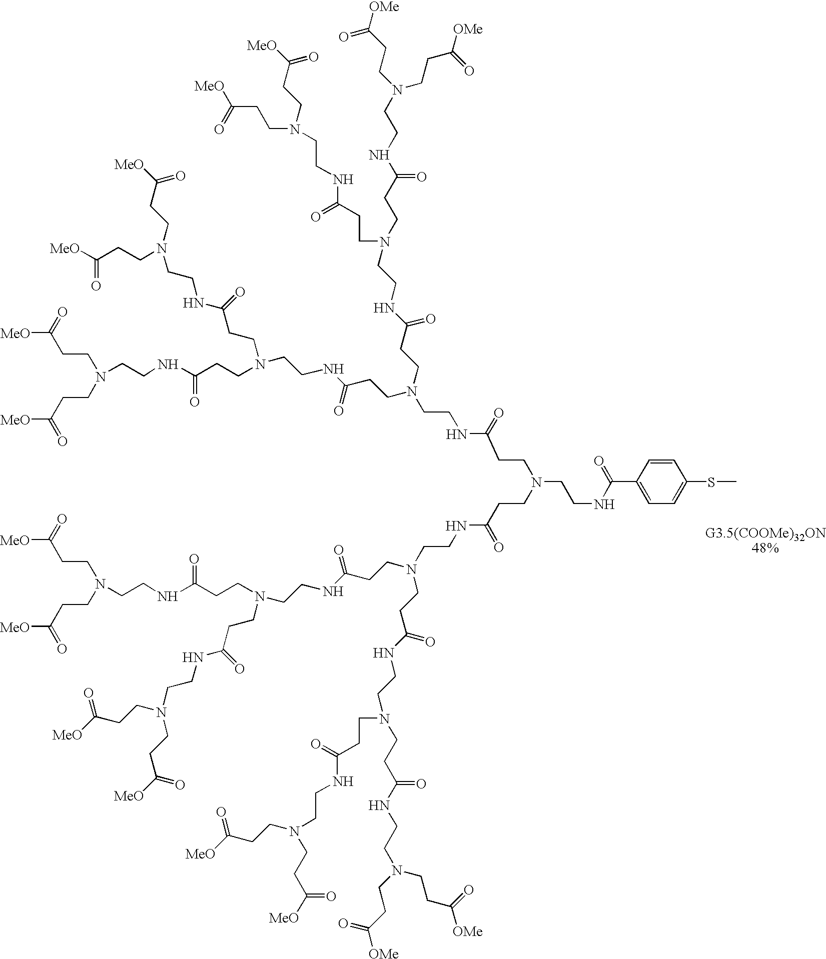 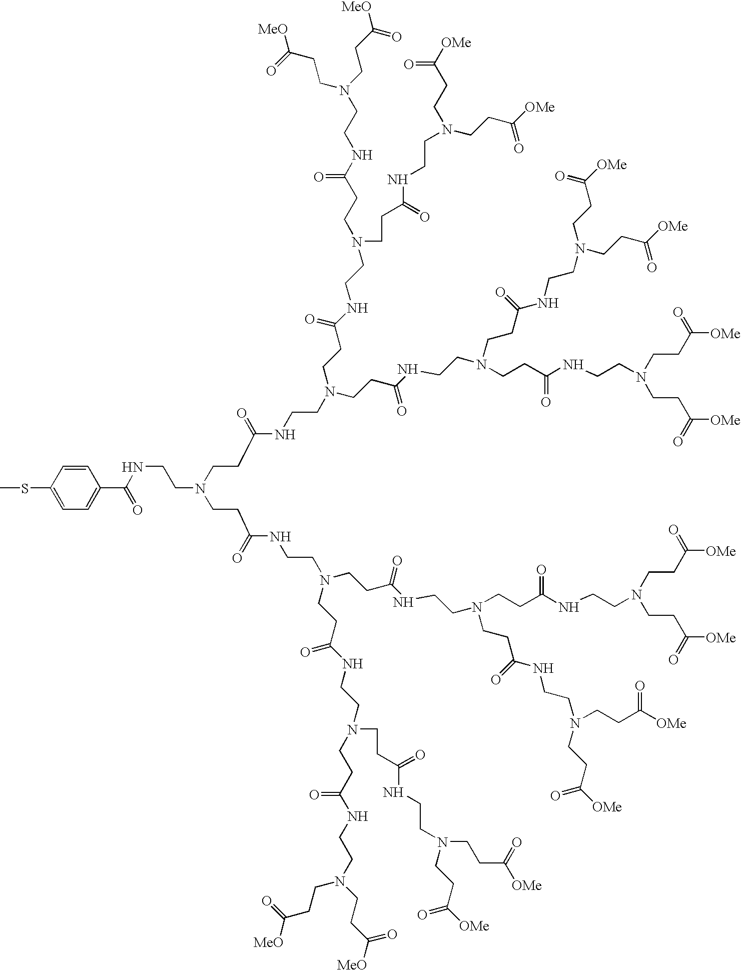
4. Synthesis of Fullerodendrimers in which C₆₀ is Incorporated into Disulfides having Dendrimer Substituents.

Generation 1.5 polyamidoaminedendrimer disulfide (G1.5 (COOMe)₈ON) and C₆₀ fullerene were photo-reacted to synthesize a new generation 1.5 fullerodendrimer (C₆₀(G1.5)₂) (Scheme 2-7, 16% yield). The (C₆₀(G1.5)₂) structure was determined by ¹H NMR, MALDI-TOF-MASS, and UV-Vis. Similarly, generation 0.5 fullerodendrimer (C₆₀(G0.5)₂) (33% yield), generation 2.5 fullerodendrimer (C₆₀(G2.5)₂) (about a 5% yield), and generation 3.5 fullerodendrimer (C₆₀(G3.5)₂) (about a 2% yield) were synthesized.

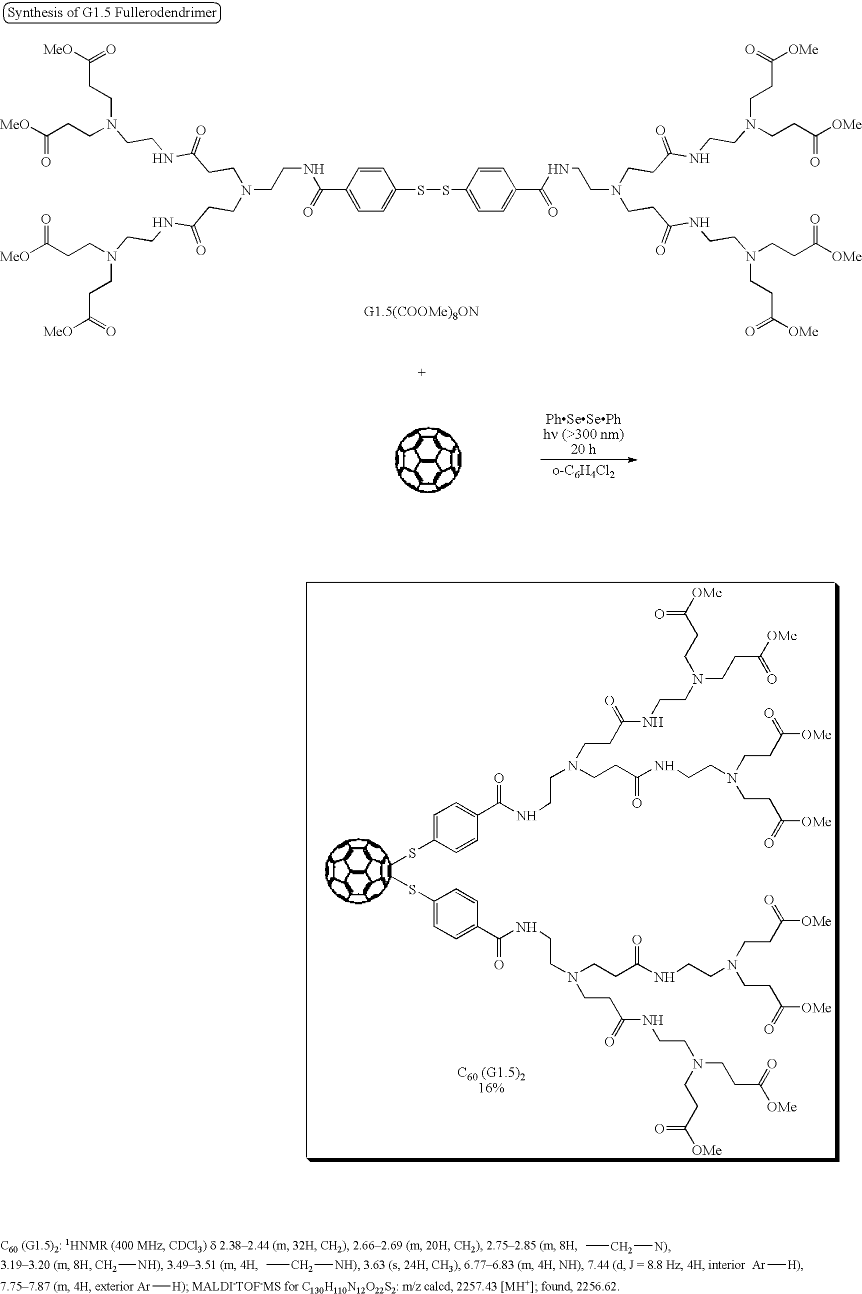

The solvents and reagents employed were purchased from Aldrich, Kanto Kagaku K.K., Tokyo Kasei Kogyo K.K., and Wako Junyaku Kogyo K.K.

The nuclear magnetic resonance (NMR) spectra were measured with a JEOL PMX60 (60 MHz) and Bruker AVANCE400 spectrometer (400 MHz). TMS was employed as the internal standard substance. Fractional high-performance liquid chromatography was conducted with a SHIMAZU CLASS-LC10 with Shodex Asahipak GF-310 HQ columns. A Japan Analytical Industry Co. Model LC-918V was employed with JAIGEL 1.0H, 2.5H, and 3.0H (eluent: CHCl₃) and JAIGEL GS-320 (eluent: MeOH) columns for analytical high-performance liquid chromatography.

A PerSeptive Biosystems Voyager Elite was employed in MALDI-TOF-MASS spectrometery. A Hitachi U-3210 was employed to determine the ultraviolet visible absorption spectrum (UV-Vis). Dynamic light scattering (DLS) was measured with a Photal DLS-7000.

### EXAMPLE 4-1

### Synthesis of 4-mercaptomethyl Benzoate (2)

4-Mercaptobenzoic acid (1) (100 g, 6.5 mmol) was suspended in CHCl₃ (4 mL). MeOH (1.74 mL, 37.7 mmol) and then concentrated sulfuric acid (1 mL) were added and the mixture was refluxed with heating overnight. The reaction solution was diluted with water and CHCl₃. The organic layer was separated and washed twice with NaHCO₃ aqueous solution. The organic layer was then dried with magnesium sulfate, filtered, concentrated, and solidified, yielding the targeted 4-mercaptomethyl benzoate (2) (1.03 g, 6.1 mmol, 95% yield) in the form of yellow crystals.

4-Mercaptomethyl benzoate (2):

¹H NMR (400 MHz, CDCl₃) δ 3.64 (s, 1H, SH), 3.88 (s, 3H, CH₃), 7.26 (d, J=8.4 Hz, 2H, interior Ar—H), 7.86 (d, J=8.4 Hz, 2H, exterior Ar—H); ¹³C NMR (100 MHz, CDCl₃) δ 51.9, 126.9, 128.0, 130.0, 138.3, 166.4.

### EXAMPLE 4-2

### Synthesis of 4,4′-dithiobismethyl Benzoate (3)

A CHCl₃ solution (100 mL) of 4-mercaptomethyl benzoate (2) (3.07 g, 18.2 mmol) and a CHCl₃ solution (100 mL) of iodine (6.86 g, 27.0 mmol) were gradually added simultaneously and dropwise with stirring to a CHCl₃ (100 mL) solution of Net₃ (3.78 mL, 27.3 mmol) and reacted for three hours at room temperature. The reaction solution was washed three times with a saturated Na₂S₂O₃ aqueous solution, dried with magnesium sulfate, filtered, concentrated, and solidified. The crude composition obtained was refined by separation by column chromatography (silica gel, eluent: CHCl₃) and recrystallization from benzene/methanol, yielding the targeted 4,4′-dithiobismethyl benzoate (3) (2.86 g, 8.54 mmol, 94% yield) in the form of white acicular crystals.

4,4-Dithiobismethyl benzoate (3):

¹H NMR (400 MHz, CDCl₃) δ (3.90 (s, 6H, CH₃), 7.52 (d, J=8.4 Hz, 4H, interior Ar—H), 7.96 (d, J=8.4 Hz, 4H, exterior Ar—H); ¹³C NMR (100 MHz, CDCl₃) δ 52.2, 126.0, 128.9, 130.3, 142.1, 166.4.

### EXAMPLE 4-3

### Synthesis of Generation 0.0 Polyamidoaminedendrimer Disulfide (G0.0(NH₂)₂ON)

4,4′-Dithiobismethyl benzoate (3) (2.0 g, 5.98 mmol) was suspended in MeOH (50 mL) and ethyleneamine (100 mL, 1.50 mol) was added dropwise with ice cooling. After reacting overnight at room temperature, the reaction solution was dried under vacuum, washed twice with diethylether, and filtered, yielding a crude product of the targeted generation 0.0 polyamidoaminedendrimer disulfide (G0.0(NH₂)₂ON) (2.85 g, 7.30 mmol, 100% yield) in the form of a yellow solid.

### EXAMPLE 4-4

### Synthesis of Generation 0.5 Polyamidoaminedendrimer Disulfide (G0.5(COOMe)₄ON)

To generation 0.0 polyamidoaminedendrimer disulfide (G0.0(NH₂)₂ON) (0.37 g, 0.947 mmol) was added MeOH (20 mL) followed by methyl acrylate (50 mL, 555 mmol) and the mixture was reacted for three days at 45° C. The reaction solution was concentrated and dried. The crude product was separated by column chromatography (silica gel, eluent: CHCl₃/MeOH=30/1) and then refined by fractional high-performance liquid chromatography, yielding the targeted generation 0.5 polyamidoaminedendrimer disulfide (G0.5(COOMe)₄ON)(0.49 g, 0.665 mmol, 70% yield) in the form of an oily yellow substance.

Generation 0.5 polyamidoaminedendrimer disulfide (G0.5(COOMe)₄ON):

¹H NMR (400 MHz, CDCl₃) δ 2.43 (t, J=6.4 Hz, 8H, —CH₂—C) 2.62 (t, J=5.6 Hz, 4H, —CH₂—N), 2.75 (t, J=6.4 Hz, 8H, —CH₂—N), 3.53 (s, 12H, CH₃), 3.55 (q, J=5.6 Hz, 4H, —CH₂—NH), 7.20 (t, J=5.6 Hz, 2H, NH), 7.51 (d, J=8.8 Hz, 4H, interior Ar—H), 7.84 (d, J=8.8 Hz, 4H, exterior Ar—H); ¹³C NMR (100 MHz, CDCl₃) δ 32.5, 37.2, 48.7, 51.4, 52.7, 126.4, 128.0, 133.5, 139.9, 166.2, 173.0.

### EXAMPLE 4-5

### Synthesis of Generation 1.0 Polyamidoaminedendrimer Disulfide (G1.0(NH₄ON)

An MeOH (100 mL) solution of generation 0.5 polyamidoaminedendrimer disulfide (G0.5(COOMe)₄ON) (3.21 g, 4.37 mmol) was added dropwise to ethyleneamine (200 mL, 3.00 mol) with ice cooling. After reacting overnight at room temperature, the reaction solution was dried under vacuum, washed twice with diethylether, and filtered, yielding a crude product of the targeted generation 1.0 polyamidoaminedendrimer disulfide (G1.0(NH₂)₄ON) (3.79 g, 4.47 mmol, 100% yield) in the form of an oily substance.

### EXAMPLE 4-6

### Synthesis of Generation 1.5 Polyamidoaminedendrimer Disulfide (G1.5(COOMe)₈ON)

Methyl acrylate (2.86 mL, 31.7 mmol) was added to an MeOH (10 mL) solution of generation 1.0 polyamidoaminedendrimer disulfide (G1.0(NH₂)₄ON) (0.38 g, 0.452 mmol) and the mixture was reacted for four days at 45° C. The reaction solution was concentrated and dried, a crude product was separated by column chromatography (silica gel, eluent: CHCl₃/MeOH=15/1, 10/1), and the product was refined by fractional high-performance liquid chromatography, yielding the targeted generation 1.5 polyamidoaminedendrimer disulfide (G1.5(COOMe)₈ON) (0.64 g, 0.4 mmol, 92% yield) in the form of an oily yellow substance.

Generation 1.5 polyamidoaminedendrimer disulfide (G1.5(COOMe)₈ON):

¹H NMR (400 MHz, CDCl₃) δ 2.33-2.42 (m, 32H, CH₂), 2.63-2.68 (m, 20H, CH₂), 2.79 (t, J=6.4 Hz, 8H, —CH₂—N), 3.18 (q, J=5.5 Hz, 8H, —CH₂—NH), 3.55 (q, J=5.3 Hz, 4H, —CH₂—NH), 3.63 (s, 24H, CH₃), 6.83 (t, J=5.5 Hz, 4H, NH), 7.49 (d, J=8.8 Hz, 4H, interior Ar—H), 7.83 (t, J=5.3 Hz, 2H, NH), 7.91 (d, J=8.8 Hz, 4H, exterior Ar—H); ¹³C NMR (100 MHz, CDCl₃) δ 32.6, 33.7, 37.0, 37.6, 49.1, 49.2, 51.5, 52.5, 52.8, 126.1, 128.2, 133.6, 139.8, 166.1, 172.2, 172.9, MALDI-TOF-MASS for C₇₀H₁₁₀N₁₂O₂₂S₂: m/z calcd, 1536.83[MH⁺]; found, 1536.07.

### EXAMPLE 4-7

### Synthesis of Generation 2.0 Polyamidoaminedendrimer Disulfide (G2.0(NH₂)_{[})ON)

A MeOH (20 mL) solution of generation 1.5 polyamidoaminedendrimer disulfide (G1.5(COOMe)₈ON) (0.70 g, 0.456 mmol) was added dropwise to ethyleneamine (50 mL, 749 mmol) with ice cooling. After reacting overnight at room temperature, the reaction solution was dried under vacuum, washed twice with diethylether, and filtered, yielding a crude product of the targeted generation 2.0 polyamidoaminedendrimer disulfide (G2.0(NH₂)₈ON) (0.74 g, 0.420 mmol, 92% yield) in the form of an oily yellow substance.

### EXAMPLE 4-8

### Synthesis of Generation 2.5 Polyamidoaminedendrimer Disulfide (G2.5(COOMe)₁₆ON)

Methyl acrylate (12.8 mL, 157 mmol) was added to a MeOH (20 mL) solution of generation 2.0 polyamidoaminedendrimer disulfide (G2.0(NH₂)₈ON) 1.04 g, 0.589 mmol) and reacted for four days at 45° C. The reaction solution was concentrated and dried, a crude product was separated by column chromatography (silica gel, eluent: CHCl₃/MeOH=15/1, MeOH), and the product was refined by fractional high-performance liquid chromatography, yielding the targeted generation 2.5 polyamidoaminedendrimer disulfide (G2.5(COOMe)₁₆ON) (1.26 g, 0.401 mmol, 68% yield) in the form of an oily substance.

Generation 2.5 polyamidoaminedendrimer disulfide (G2.5(COOMe)₁₆ON):

¹H NMR (400 MHz, CDCl₃) δ 2.26-2.49 (m, 84H, CH₂), 2.62-2.76 (m, 56H, CH₂), 3.15-3.23 (m, 24H, CH₂—NH), 3.49 (q, J=5.2 Hz, 4H, CH₂—NH), 3.61 (s, 48H, CH₃), 7.03 (t, J=5.2 Hz, 8H, NH), 7.45 (d, J=8.4 Hz, 4H, interior Ar—H), 7.53 (t, J=4.8 Hz, 4H, NH), 7.86 (d, J=8.4 Hz, 4H, exterior Ar—H), 7.98 (t, J=5.2 Hz, 2H, NH); ¹³C NMR (100 MHz, CDCl₃), δ 32.6, 33.6, 33.7, 37.1, 37.3, 37.8, 49.1, 49.5, 49.6, 51.5, 52.3, 52.4, 52.8, 126.1, 128.3, 133.4, 139.8, 166.3, 172.3, 172.4, 172.9; MALDI-TOF-MASS for C₁₄₂H₂₃₈N₂₈O₄₆S₂: m/z calcd, 3160.69[MNa⁺]; found, 3160.67.

### EXAMPLE 4-9

### Synthesis of Generation 3.0 Polyamidoaminedendrimer Disulfide (G3.0(NH₂)₁₆ON)

A MeOH (15 mL) solution of generation 2.5 polyamidoaminedendrimer disulfide (G2.5(COOMe)₁₆ON) (0.21 g, 0.067 mmol) was added dropwise to ethylenediamine (18 mL, 270 mmol) with ice cooling. After reacting overnight at room temperature, the reaction solution was vacuum dried, washed twice with diethylether, and filtered, yielding a crude product of the targeted generation 3.0 polyamidoaminedendrimer disulfide (G3.0(NH₂)₁₆ON) (0.25 g, 0.070 mmol, 100% yield) in the form of an oily yellow substance.

### EXAMPLE 4-10

### Synthesis of Generation 3.5 Polyamidoaminedendrimer Disulfide (G3.5(COOMe)₃₂ON)

Methyl acrylate (4.9 mL, 53 mmol) was added to a MeOH (151 mL) solution of generation 3.0 polyamidoaminedendrimer disulfide (G3.0(NH₂)₁₆ON) (0.301 g, 0.084 mmol) and the mixture was reacted for four days at 45° C. The reaction solution was concentrated, solidified, washed three times with water/CHCl₃, and extracted. It was dried with magnesium sulfate, filtered, concentrated, and solidified. The crude product thus obtained was refined by fractional high-performance liquid chromatography, yielding the targeted generation 3.5 polyamidoaminedendrimer disulfide (G3.5(COOMe)₃₂ON) (0.25 g, 0.039 mmol, 47% yield) in the form of an oily yellow substance. However, the signals overlapped in ¹³C NMR, precluding adequate analysis.

Generation 3.5 polyamidoaminedendrimer disulfide (G3.5(COOMe)₃₂ON):

¹H NMR (400 MHz, CDCl₃) δ 2.24-2.47 (m, 180H, CH₂), 2.59-2.72 (m, 120H, CH₃), 3.17-3.20 (m, 56H, —CH₂—NH), 3.42 (brs, 4H, —CH₂—NH), 3.57 (s, 96H, CH₃), 6.99 (t, J=4.8 Hz, 16H, NH), 7.41 (d, J=8.4 Hz, 4H, interior Ar—H), 7.55 (brs, 8H, NH), 7.63 (brs, 4H, NH), 7.82 (d, J=8.4 Hz, 4H, exterior Ar—H), 8.01 (brs, 2H, NH); ¹³C NMR (100 MHz, CDCl₃) δ (32.5, 33.6, 37.0, 37.3, 49.1, 49.6, 51.4, 52.3, 52.7, 126.1, 128.2, 172.2, 172.4, 172.9; MALDI-TOF-MASS for C₂₈₆H₄₉₄N₆₀O₉₄S₂: m/z calcd, 6364.45[MNa⁺]; found, 6364.73.

### EXAMPLE 4-11

### Synthesis of Generation 0.5 Fullerodendrimer (C₆₀(G0.5)₂)

To a Pyrex test tube were charged generation 0.5 polyamidoaminedendrimer disulfide (G0.5(COOMe)₄ON) (0.300 g, 0.408 mmol), C₆₀ (0.059 mg, 0.082 mmol), diphenyl diselenide (0.013 g, 0.04 mmol). o-C₆H₄Cl₂ was added and ultrasound was applied until complete dissolution was achieved. While bubbling N₂, a high-pressure mercury lamp (≧300 nm) was employed to conduct a photo-reaction for 22 hours. The reaction solution was concentrated and solidified with a vacuum pump equipped with trap, dissolved in MeOH, and suction filtered with a Buchner. The crude product obtained was refined by partitional high-performance liquid chromatography, yielding the targeted generation 0.5 fullerodendrimer (C₆₀(G0.5)₂) (0.039 g, 0.026 mmol, 33% yield) in the form of an oily, lightly brown substance.

Generation 0.5 fullerodendrimer (C₆₀(G0.5)₂):

¹H NMR (400 MHz, CDCl₃) δ (2.42-2.46 (m, 8H, —CH₂—C), 2.62-2.65 (m, 4H, —CH₂—N), 2.71-2.78 (m, 8H, —CH₂—N), 3.53 (s, 12H, CH₃), 3.55 (q, J=5.6 Hz, 4H, —CH₂—NH), 7.12-7.14 (m, 2H, NH), 7.30 (d, J=8.8 Hz, 4H, interior Ar—H), 7.49 (d, J=8.8 Hz, 4H, exterior Ar—H); ¹³C NMR (100 MHz, CDCl₃) δ 32.5, 37.2, 48.7, 51.4, 52.7, 61.7, 62.1, 125.9, 126.5, 127.2, 127.5, 128.0, 128.7, 129.5, 129.7, 130.0, 131.5, 132.3, 132.6, 133.5, 133.9, 134.4, 135.6, 139.2, 139.8, 140.0, 166.2, 173.0; MALDI-TOF-MASS for C₉₄H₄₆N₄O₁₀S₂: m/z calcd, 1456.49[MH⁺]; found, 1455.94.

### EXAMPLE 4-12

### Synthesis of Generation 1.5 Fullerodendrimer (C₆₀(G1.5)₂)

To a Pyrex test tube were charged generation 1.5 polyamidoaminedendrimer disulfide (G1.5(COOMe)₄ON) (0.250 g, 0.027 mmol), C₆₀ (0.024 mg, 0.033 mmol), and diphenyl diselenide (0.051 g, 0.1631 mmol). o-C₆H₄Cl₂ (5 mL) was added and ultrasound was applied until complete dissolution was achieved. While bubbling N₂, a high-pressure mercury lamp (≧300 nm) was employed to conduct a photo-reaction for 20 hours. The reaction solution was concentrated and solidified with a vacuum pump equipped with trap, dissolved in CHCl₃, and suction filtered with a Buchner. The crude product obtained was refined by partitional high-performance chromatography, yielding the targeted generation 1.5 fullerodendrimer (C₆₀(G1.5)₂) (0.012 g, 0.005 mmol, 16% yield) in the form of an oily, brown substance. Absorption was observed at 432.8 nm and 704.2 nm by UV-Vis, matching the literature. However, the signal became broad in ¹H NMR and ¹³C NMR, precluding adequate analysis.

Generation 1.5 fullerodendrimer (C₆₀(G1.5)₂):

¹H NMR (400 MHz, CDCl₃) δ 2.38-2.44 (m, 32H, CH₂), 2.66-2.69 (m, 20H, CH₂), 2.80-2.82 (m, 8H, —CH₂—N), 3.19-3.20 (m, 8H, —CH₂—NH), 3.49-3.52 (m, 4H, —CH₂—NH), 3.64 (s, 24H, CH₃), 6.77-6.83 (m, 4H, NH), 7.43 (d, J=7.2 Hz, 4H, interior Ar—H), 7.75-7.77 (m, 2H, NH), 7.85 (d, J=7.2 Hz, 4H, exterior Ar—H); ¹³C NMR (100 MHz, CDCl₃) δ 32.6, 33.7, 37.0, 37.6, 49.1, 49.2, 51.5, 52.5, 52.8, 58.4, 70.6, 126.1, 126.2, 127.7, 128.0-128.2, 128.3, 129.1-129.9, 130.5, 132.6, 133.7, 139.9, 166.2, 172.3, 173.0; MALDI-TOF-MASS for C₁₃₀H₁₁₀N₁₂O₂₂S₂: m/z calcd, 2257.47[MH⁺]; found, 2256.62.

### EXAMPLE 4-13

### Synthesizing Generation 2.5 Fullerodendrimer (C₆₀(G2.5)₂)

To a Pyrex test tube were charged generation 2.5 polyamidoaminedendrimer disulfide (G2.5(COOMe)₄ON) (0.200 g, 0.064 mmol), C₆₀ (0.023 mg, 0.032 mmol), and diphenyl diselenide (0.020 g, 0.064 mmol). o-C₆H₄Cl₂ (2 mL) was added and ultrasound was applied until complete dissolution was achieved. While bubbling N₂, a high-pressure mercury lamp (≧300 nm) was employed to conduct a photo-reaction for 3 hours. The reaction solution was concentrated and solidified with a vacuum pump equipped with trap, dissolved in MeOH, and suction filtered with a Buchner. The crude product obtained was refined by partitional high-performance liquid chromatography, yielding the targeted generation 2.5 fullerodendrimer (C₆₀(G2.5)₂) (0.006 g, 0.002 mmol, 5% yield) in the form of an oily, brown substance. Absorption was observed at 432.81 nm and 704.2 nm by UV-Vis, matching the literature. However, determination was not possible by ¹H NMR, ¹³C NMR, or MALDI-TOF-MASS spectrometry.

### EXAMPLE 4-14

### Synthesizing Generation 3.5 Fullerodendrimer (C₆₀(G3.5)₂)

To a Pyrex test tube were charged generation 3.5 polyamidoaminedendrimer disulfide (G3.5(COOMe)₄ON) (0.100 g, 0.016 mmol), C₆₀ (0.012 mg, 0.016 mmol), and diphenyl diselenide (0.005 g, 0.016 mmol). o-C₆H₄Cl₂ (10 mL) was added and ultrasound was applied until complete dissolution was achieved. While bubbling N₂, a high-pressure mercury lamp (≧300 nm) was employed to conduct a photo-reaction for 3 hours. The reaction solution was concentrated and solidified with a vacuum pump equipped with trap, dissolved in MeOH, and suction filtered with a Buchner. The crude product obtained was refined by partitional high-performance liquid chromatography, yielding a product like generation 3.5 fullerodendrimer (C₆₀(G3.5)₂) (0.002 g, 0.283 micromol, 2% yield) in the form of an oily, brown substance with a greater molecular weight than the starting material (G3.5(COOMe)₄ON) based on GPC retention time. However, the structure of the substance could not be determined by ¹H NMR, ¹³C NMR, or MALDI-TOF-MASS spectrometry.

Determination of the Structure of Generation 1.5 Fullerodendrimer (C₆₀(G1.5)₂) by UV-vis Spectrometry

When the Uv-vis spectrum of an o-C₆H₄Cl₂ solution of (C₆₀(G1.5)₂) (0.13 mmol/L) was measured, absorption was observed at 432.8 nm and 704.2 nm. These values were confirmed to be roughly identical to the values in the literature for 6-6′ added fullerene derivatives.

Determination of the Structure of Generation 1.5 Fullerodendrimer (C₆₀(G1.5)₂) by MALDI-TOF-MS

When 9-nitroanthracene was added as matrix to generation 1.5 fullerodendrimer and measurement was conducted in negative mode with a laser intensity of 4,600, the parent peak was confirmed to match the numerical value of the molecular weight of the targeted substance. Of considerable further interest, since (C₆₀(G1.5)₁) to which a dendron had been added at 1488.09 was confirmed, it was found that the C—S bond of the adduct was severed at high energy.

### EXAMPLE 5

### The Stability of Fullerodendrimer

When 1.5 fullerodendrimer (C₆₀(G1.5)₂) was dissolved in a mixed solvent of CHCl₃/MeOH=1/10 and refluxed for 14 days at 70° C., the destruction of the dendrimer was confirmed in some cases, but there was no cleavage of the C—S bond resulting in the release of C₆₀. Subsequently, the solution was transferred to a Pyrex threaded-neck test tube and irradiated with a high-pressure mercury lamp (≧300 nm) for three days, but severing of the C—S bond could not be confirmed. Similar results were achieved for 0.5 and generation 2.5 fullerodendrimers. This clearly showed that the C—S bond possessed high heat stability.

### EXAMPLE 6

### The Oxidation and Reduction Potential of Fullerodendrimer

Measurement of the oxidation and reduction potential of (C₆₀(G0.5)₂) and (C₆₀(G1.5)₂) revealed the first oxidation potential of the two to be +732 mV and +766 mV, respectively, and the first reduction potential of the two to be −1,100 mV and −1,084 mV, respectively. This revealed that, compared to first oxidation and reduction potentials of C₆₀ of +1,120 mV and −1,120 mV, (C₆₀(G0.5)₂) and (C₆₀(G1.5)₂) had a much stronger tendency to oxidize and a much weaker tendency to undergo reduction than C₆₀. The addition of sulfur to C₆₀ was found to further increase donor properties.

### EXAMPLE 7

### Examination of the Solubility in MeOH of Fullerodendrimers

(C₆₀(G0.5)₂), (C₆₀(G1.5)₂), and (C₆₀(G2.5)₂) were dissolved ((C₆₀(G0.5)₂) and (C₆₀(G1.5)₂) were dispersed) in 1 mL of MeOH and passed through a membrane filter (200 micrometers), giving results of (4 mL, 2.7 micromols) for (C₆₀(G0.5)₂), (9 mL, 3.9 micromols) for (C₆₀(G1.5)₂), and (>50 mL, >12.9 micromols) for (C₆₀(G2.5)₂). This clearly showed that solubility increased with the addition of terminal substituents on the dendrimers.

### EXAMPLE 8

### Examination of the Solubility in Water of Fullerodendrimers

When (C₆₀(G0.5)₂) and (C₆₀(G1.5)₂) were dissolved in pH 3 water, a brown solution was obtained. Since these substances would not dissolve in pH 7 water, it was thought that water solubility resulted from protonation of the tertiary amines in the polyamidoaminedendrimer skeleton under acidic conditions.

### EXAMPLE 9

### Measurement by DLS (Dynamic Light Scattering)

A CHCl₃ solution of (C₆₀(G0.5)₂) (10.8 mmol/L) was diffused by ultrasound and passed through a 200 nm membrane filter. Measurement of the particle diameters (500 particle summation) revealed an estimated particle diameter of 45.9 (±0.2) nm. When an MeOH solution of (C₆₀(G0.5)₂) (30.9 mmol/L) was diffused by ultrasound, an estimated particle diameter of 458.1 nm was measured after 5 min, an estimated particle diameter of 1,920.5 nm was measured after 15 min, and an estimated particle diameter of 5,287.4 nm was measured after 30 min, with the (C₆₀(G0.5)₂) appearing as a precipitate visible to the eye. This clearly indicated that the fullerodendrimer assumed the form of a molecular aggregate, having a tendency to aggregate that increased with the polarity of the solvent and assuming a state of high molecular aggregation.

### EXAMPLE 10

The fullerodendrimers of above-described Example 2-1 to 2-10 and Examples 4-1 to 4-14 were used to prepare 0.01M chloroform solutions. These solutions were spin coated to form thin layers containing fullerodendrimers.

Measurement by Atomic Force Microscopy (AFM)

The thin films obtained were observed by AFM, revealing that the fullerodendrimer aggregates had relatively uniformly aggregated, with a width of about 200 nm and a height of about 4 nm.

### EXAMPLE 11

Mixtures of the compositions given below were prepared from the fullerodendrimers of above-described Example 2-1 to 2-10 and Examples 4-1 to 4-14 and shaken for 3 hours in a paint shaker to achieve thorough mixing and dispersion, yielding paint compositions. The Lumiflon LF200C referred to below is a fluoropolymer comprised chiefly of a copolymer of vinyl ether and fluoroolefin.

A paint composition comprising 6.2 g of fullerodendrimer, 0.80 g of fluoropolymer (Lumiflon LF200C made by Asahi Glass), 0.16 g of isocyanate hardener, 1.00 g of titanium coupling agent (Plain Act 338X made by Ajinomoto), and 23.60 mL of toluene was applied to a 20 cm² plate of glass and dried for 20 min at a temperature of 120° C. to obtain the coating film of the present invention.

1. A fullerodendrimer denoted by general formula (1) or (2): 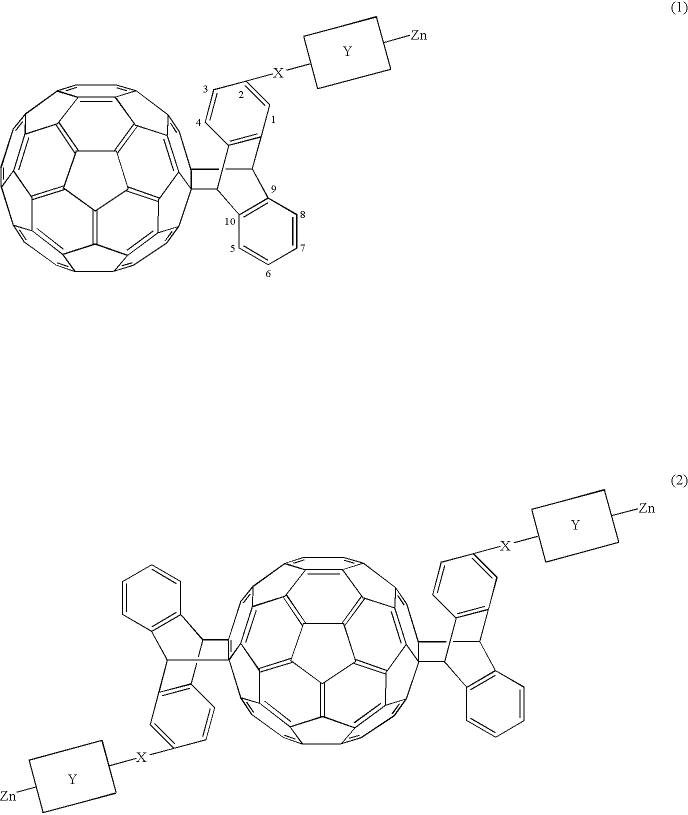
wherein X denotes an electron-attracting substituent, Y denotes a spacer, and Z denotes a terminal functional group required to achieve a function, with the number n of Z incorporated in Y being from 1 to 3.

2. The fullerodendrimer according to claim 1, wherein the fullerene is C₆₀ or C₇₀.

3. A thin film comprising the fullerodendrimer according to claim 1 or 2.

4. The thin film according to claim 3 further comprising an organic polymer and/or an inorganic polymer.

5. A fullerodendrimer denoted by general formula (3): 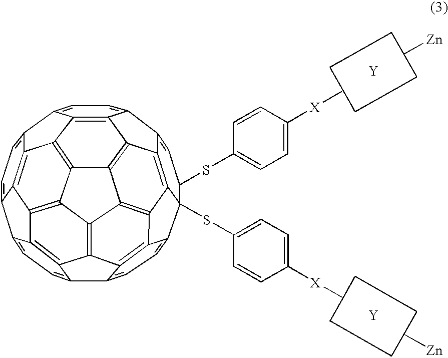
wherein X denotes an electron-attracting substituent, Y denotes a spacer, and Z denotes a terminal functional group required to achieve a function, with the number n of Z incorporated in Y being from 1 to 3.

6. The fullerodendrimer according to claim 5, wherein the fullerene is C₆₀ or C₇₀.

7. A thin film comprising the fullerodendrimer according to claim 5 or 6.

8. The thin film according to claim 7 further comprising an organic polymer and/or an inorganic polymer.

9. A method of manufacturing a thin film comprising the fullerodendrimer denoted by any of general formulas (1) to (3), comprising the coating on a substrate of a mixture of any of the fullerodendrimers denoted by any of general formulas (1) to (3) with a solvent.

10. A method of manufacturing a thin film comprising any of the fullerodendrimers denoted by any of general formulas (1) to (3) and an organic polymer and/or inorganic polymer, comprising coating on a substrate of a mixture of any of the fullerodendrimers denoted by any of general formulas (1) to (3), an organic polymer and/or inorganic polymer, and a solvent.

11. A product comprising a substrate and a fullerene thin film provided on said substrate.

12. A method of manufacturing a product comprising a substrate on which is provided a fullerene thin film, comprising providing on a substrate of the thin film according to claim 3 and decomposing of at least the dendrimer constituting the fullerodendrimer by heating in a non-oxidizing atmosphere.

13. A method of manufacturing a product comprising a substrate on which is provided a fullerene thin film, comprising providing on a substrate of the thin film according to claim 7 and decomposing of at least the dendrimer constituting the fullerodendrimer by heating in a non-oxidizing atmosphere.
